SEQUENCE LISTING (311 sequences)

SEQ ID NO: 1 - protein, 9 aa, synthetic construct
  source 1..9: note = 164
AQLHGLRVL

SEQ ID NO: 2 - protein, 9 aa, synthetic construct
  source 1..9: note = 165
AQRQGLRLL

SEQ ID NO: 3 - protein, 9 aa, synthetic construct
  source 1..9: note = 166
AQMRRGTTN

SEQ ID NO: 4 - protein, 9 aa, synthetic construct
  source 1..9: note = 167
AQLGRRVMV

SEQ ID NO: 5 - protein, 9 aa, synthetic construct
  source 1..9: note = 168
AQRLLRIEK

SEQ ID NO: 6 - protein, 9 aa, synthetic construct
  source 1..9: note = 169
AQFGKVWKV

SEQ ID NO: 7 - protein, 9 aa, synthetic construct
  source 1..9: note = 170
AQVGRRALP

SEQ ID NO: 8 - protein, 9 aa, synthetic construct
  source 1..9: note = 171
AQRFGGRGA

SEQ ID NO: 9 - protein, 9 aa, synthetic construct
  source 1..9: note = 172
AQQPRLRVG

SEQ ID NO: 10 - protein, 9 aa, synthetic construct
  source 1..9: note = 173
AQSTSRTRD

SEQ ID NO: 11 - protein, 9 aa, synthetic construct
  source 1..9: note = 174
AQRSRGSYP

SEQ ID NO: 12 - protein, 9 aa, synthetic construct
  source 1..9: note = 175
AQVAWGQRL

SEQ ID NO: 13 - protein, 9 aa, synthetic construct
  source 1..9: note = 176
DGPLGVYRR

SEQ ID NO: 14 - protein, 9 aa, synthetic construct
  source 1..9: note = 177
AQRSAIVRM

SEQ ID NO: 15 - protein, 9 aa, synthetic construct
  source 1..9: note = 178
DGPPTWRSR

SEQ ID NO: 16 - protein, 9 aa, synthetic construct
  source 1..9: note = 179
DGPSLKSPR

SEQ ID NO: 17 - protein, 9 aa, synthetic construct
  source 1..9: note = 180
AQRLPLTRA

SEQ ID NO: 18 - protein, 9 aa, synthetic construct
  source 1..9: note = 181
AQVGRYKVS

SEQ ID NO: 19 - protein, 9 aa, synthetic construct
  source 1..9: note = 182
AQRRPILLG

SEQ ID NO: 20 - protein, 9 aa, synthetic construct
  source 1..9: note = 183
DGPNPYRLR

SEQ ID NO: 21 - protein, 9 aa, synthetic construct
  source 1..9: note = 184
DGPARLSIR

SEQ ID NO: 22 - protein, 9 aa, synthetic construct
  source 1..9: note = 185
DGPLRMPNR

SEQ ID NO: 23 - protein, 9 aa, synthetic construct
  source 1..9: note = 186
AQRSTLVRS

SEQ ID NO: 24 - protein, 9 aa, synthetic construct
  source 1..9: note = 187
DGPGPGVSR

SEQ ID NO: 25 - protein, 9 aa, synthetic construct
  source 1..9: note = 188
DGPRKSTYR

SEQ ID NO: 26 - protein, 9 aa, synthetic construct
  source 1..9: note = 189
DGPWVNSPR

SEQ ID NO: 27 - protein, 9 aa, synthetic construct
  source 1..9: note = 190
DGPRPYATR

SEQ ID NO: 28 - protein, 9 aa, synthetic construct
  source 1..9: note = 191
DGPWVNTPR

SEQ ID NO: 29 - protein, 9 aa, synthetic construct
  source 1..9: note = 192
AQSRARPAL

SEQ ID NO: 30 - protein, 9 aa, synthetic construct
  source 1..9: note = 193
AQGRSEQSP

SEQ ID NO: 31 - protein, 9 aa, synthetic construct
  source 1..9: note = 194
DGPYSRTTR

SEQ ID NO: 32 - protein, 9 aa, synthetic construct
  source 1..9: note = X- 256-1
AQVMRLSAL

SEQ ID NO: 33 - protein, 9 aa, synthetic construct
  source 1..9: note = X-257-2
AQVNGLRVL

SEQ ID NO: 34 - protein, 9 aa, synthetic construct
  source 1..9: note = X-258-3
AQRMINAAW

SEQ ID NO: 35 - protein, 9 aa, synthetic construct
  source 1..9: note = X-259-4
AQREGLRLL

SEQ ID NO: 36 - protein, 9 aa, synthetic construct
  source 1..9: note = X-260-5
AQRQRLEVL

SEQ ID NO: 37 - protein, 9 aa, synthetic construct
  source 1..9: note = 2nd-Camp#6
AQRMRLEIL

SEQ ID NO: 38 - protein, 9 aa, synthetic construct
  source 1..9: note = 2nd-Camp#7
AQAPSFRSS

SEQ ID NO: 39 - protein, 9 aa, synthetic construct
  source 1..9: note = 2nd-Camp#8
AQKVHVPQW

SEQ ID NO: 40 - protein, 9 aa, synthetic construct
  source 1..9: note = 2nd-Camp#9
AQRGGLSVL

SEQ ID NO: 41 - protein, 9 aa, synthetic construct
  source 1..9: note = 2nd-Camp#10
AQIRGISVI

SEQ ID NO: 42 - protein, 9 aa, synthetic construct
  source 1..9: note = 2nd-Camp#11
AQFRGLSSL

SEQ ID NO: 43 - protein, 9 aa, synthetic construct
  source 1..9: note = 2nd-Camp#12
AQYSQTWRV

SEQ ID NO: 44 - protein, 9 aa, synthetic construct
  source 1..9: note = 2nd-Camp#13
AQAASWRSA

SEQ ID NO: 45 - protein, 9 aa, synthetic construct
  source 1..9: note = 2nd-Camp#14
AQMEGSWRV

SEQ ID NO: 46 - protein, 9 aa, synthetic construct
  source 1..9: note = 2nd-Camp#15
AQRLALRTL

SEQ ID NO: 47 - protein, 9 aa, synthetic construct
  source 1..9: note = 2nd-Camp#16
AQRMGLSSL

SEQ ID NO: 48 - protein, 9 aa, synthetic construct
  source 1..9: note = 2nd-Camp#17
AQFRQLSVL

SEQ ID NO: 49 - protein, 9 aa, synthetic construct
  source 1..9: note = 2nd-Camp#18
AQFRGSWSV

SEQ ID NO: 50 - protein, 9 aa, synthetic construct
  source 1..9: note = 2nd-Camp#19
AQFKPSWSV

SEQ ID NO: 51 - protein, 9 aa, synthetic construct
  source 1..9: note = 2nd-Camp#20
AQMKPIWAV

SEQ ID NO: 52 - protein, 9 aa, synthetic construct
  source 1..9: note = 2nd-Camp#21
AQLMRLSLL

SEQ ID NO: 53 - protein, 9 aa, synthetic construct
  source 1..9: note = 2nd-Camp#22
AQTLYVNKW

SEQ ID NO: 54 - protein, 9 aa, synthetic construct
  source 1..9: note = 2nd-Camp#23
AQRNALRSL

SEQ ID NO: 55 - protein, 9 aa, synthetic construct
  source 1..9: note = 2nd-Camp#24
AQSVTVPRW

SEQ ID NO: 56 - protein, 9 aa, synthetic construct
  source 1..9: note = 2nd-Camp#25
AQRLANPRW

SEQ ID NO: 57 - protein, 9 aa, synthetic construct
  source 1..9: note = 2nd-Camp#26
AQRARVAVA

SEQ ID NO: 58 - protein, 9 aa, synthetic construct
  source 1..9: note = X-261 #27
AQIRGLRVL

SEQ ID NO: 59 - protein, 9 aa, synthetic construct
  source 1..9: note = 2nd-Camp#28
AQLTPRWHV

SEQ ID NO: 60 - protein, 9 aa, synthetic construct
  source 1..9: note = 2nd-Camp#29
AQHVTMSRW

SEQ ID NO: 61 - protein, 9 aa, synthetic construct
  source 1..9: note = 2nd-Camp#30
AQMGSVWRV

SEQ ID NO: 62 - protein, 9 aa, synthetic construct
  source 1..9: note = 2nd-Camp#31
AQVLGYGHW

SEQ ID NO: 63 - protein, 9 aa, synthetic construct
  source 1..9: note = X-262 #32
AQSMGLRVL

SEQ ID NO: 64 - protein, 9 aa, synthetic construct
  source 1..9: note = 2nd-Camp#33
DGPNMVYSR

SEQ ID NO: 65 - protein, 9 aa, synthetic construct
  source 1..9: note = 2nd-Camp#34
AQNALAFAT

SEQ ID NO: 66 - protein, 9 aa, synthetic construct
  source 1..9: note = 2nd-Camp#35
AQKRMSYPA

SEQ ID NO: 67 - protein, 9 aa, synthetic construct
  source 1..9: note = 2nd-Camp#36
AQYRALSTL

SEQ ID NO: 68 - protein, 9 aa, synthetic construct
  source 1..9: note = 2nd-Camp#37
AQTVYTARW

SEQ ID NO: 69 - protein, 9 aa, synthetic construct
  source 1..9: note = 2nd-Camp#38
AQWGQRWHV

SEQ ID NO: 70 - protein, 9 aa, synthetic construct
  source 1..9: note = 2nd-Camp#39
AQRHPVGLG

SEQ ID NO: 71 - protein, 9 aa, synthetic construct
  source 1..9: note = 2nd-Camp#40
AQRRSSPST

SEQ ID NO: 72 - protein, 9 aa, synthetic construct
  source 1..9: note = 2nd-Camp#41
AQWNARWHV

SEQ ID NO: 73 - protein, 9 aa, synthetic construct
  source 1..9: note = 2nd-Camp#42
AQFSARWSV

SEQ ID NO: 74 - protein, 9 aa, synthetic construct
  source 1..9: note = 2nd-Camp#43
AQERGRSSR

SEQ ID NO: 75 - protein, 9 aa, synthetic construct
  source 1..9: note = 2nd-Camp#44
AQRLPARVS

SEQ ID NO: 76 - protein, 9 aa, synthetic construct
  source 1..9: note = 2nd-Camp#45
AQTLRLRSR

SEQ ID NO: 77 - protein, 9 aa, synthetic construct
  source 1..9: note = 2nd-Camp#46
AQPRSKISL

SEQ ID NO: 78 - protein, 9 aa, synthetic construct
  source 1..9: note = 2nd-Camp#47
AQFPRVRRR

SEQ ID NO: 79 - protein, 9 aa, synthetic construct
  source 1..9: note = 2nd-Camp#48
AQHRGSRVP

SEQ ID NO: 80 - protein, 9 aa, synthetic construct
  source 1..9: note = 2nd-Camp#49
AQMMTRRHV

SEQ ID NO: 81 - protein, 9 aa, synthetic construct
  source 1..9: note = 2nd-Camp#50
AQHPLRRVW

SEQ ID NO: 82 - protein, 9 aa, synthetic construct
  source 1..9: note = 2nd-Camp#51
AQGSKLLGR

SEQ ID NO: 83 - protein, 9 aa, synthetic construct
  source 1..9: note = 2nd-Camp#52
DGPYLGLFR

SEQ ID NO: 84 - protein, 9 aa, synthetic construct
  source 1..9: note = 2nd-Camp#53
AQSYPHARG

SEQ ID NO: 85 - protein, 9 aa, synthetic construct
  source 1..9: note = 2nd-Camp#54
AQWHEFHSP

SEQ ID NO: 86 - protein, 9 aa, synthetic construct
  source 1..9: note = 2nd-Camp#55
AQRQNRGPL

SEQ ID NO: 87 - protein, 9 aa, synthetic construct
  source 1..9: note = 2nd-Camp#56
AQWSNKWHV

SEQ ID NO: 88 - protein, 9 aa, synthetic construct
  source 1..9: note = 2nd-Camp#57
AQRGAINFR

SEQ ID NO: 89 - protein, 9 aa, synthetic construct
  source 1..9: note = 2nd-Camp#58
AQRTYSVSA

SEQ ID NO: 90 - protein, 9 aa, synthetic construct
  source 1..9: note = 2nd-Camp#59
AQLLHRNRR

SEQ ID NO: 91 - protein, 9 aa, synthetic construct
  source 1..9: note = 2nd-Camp#60
AQWAFQGRG

SEQ ID NO: 92 - protein, 9 aa, synthetic construct
  source 1..9: note = 2nd-Camp#61
AQRASLRTL

SEQ ID NO: 93 - protein, 9 aa, synthetic construct
  source 1..9: note = 2nd-Camp#62
AQRSLHSRT

SEQ ID NO: 94 - protein, 9 aa, synthetic construct
  source 1..9: note = 2nd-Camp#63
AQPKRPVAI

SEQ ID NO: 95 - protein, 9 aa, synthetic construct
  source 1..9: note = 2nd-Camp#64
AQHTFRSVA

SEQ ID NO: 96 - protein, 9 aa, synthetic construct
  source 1..9: note = 2nd-Camp#65
DGPVVRSRR

SEQ ID NO: 97 - protein, 9 aa, synthetic construct
  source 1..9: note = X-263 #66
AQDRGLRVL

SEQ ID NO: 98 - protein, 9 aa, synthetic construct
  source 1..9: note = 2nd-Camp#67
DGPHFSRTR

SEQ ID NO: 99 - protein, 9 aa, synthetic construct
  source 1..9: note = 2nd-Camp#68
AQLRPVKLL

SEQ ID NO: 100 - protein, 9 aa, synthetic construct
  source 1..9: note = 2nd-Camp#69
AQFRPVPRA

SEQ ID NO: 101 - protein, 9 aa, synthetic construct
  source 1..9: note = 2nd-Camp#70
AQLNVVRRV

SEQ ID NO: 102 - protein, 9 aa, synthetic construct
  source 1..9: note = 2nd-Camp#71
AQRIARPPF

SEQ ID NO: 103 - protein, 9 aa, synthetic construct
  source 1..9: note = 2nd-Camp#72
AQASAMKRP

SEQ ID NO: 104 - protein, 9 aa, synthetic construct
  source 1..9: note = 2nd-Camp#73
AQVRPSVKA

SEQ ID NO: 105 - protein, 9 aa, synthetic construct
  source 1..9: note = 2nd-Camp#74
AQDRKQRPS

SEQ ID NO: 106 - protein, 9 aa, synthetic construct
  source 1..9: note = 2nd-Camp#75
AQKNVPKAS

SEQ ID NO: 107 - protein, 9 aa, synthetic construct
  source 1..9: note = 2nd-Camp#76
AQGLSRWAP

SEQ ID NO: 108 - protein, 9 aa, synthetic construct
  source 1..9: note = 2nd-Camp#77
AQFKHKGLI

SEQ ID NO: 109 - protein, 9 aa, synthetic construct
  source 1..9: note = 2nd-Camp#78
AQRLALPRS

SEQ ID NO: 110 - protein, 9 aa, synthetic construct
  source 1..9: note = 2nd-Camp#79
AQRPPRRSG

SEQ ID NO: 111 - protein, 9 aa, synthetic construct
  source 1..9: note = 2nd-Camp#80
AQTRGPWRV

SEQ ID NO: 112 - protein, 9 aa, synthetic construct
  source 1..9: note = 2nd-Camp#81
DGPTIRYLR

SEQ ID NO: 113 - protein, 9 aa, synthetic construct
  source 1..9: note = 2nd-Camp#82
AQPRRTRVA

SEQ ID NO: 114 - protein, 9 aa, synthetic construct
  source 1..9: note = 2nd-Camp#83
AQSRASRVS

SEQ ID NO: 115 - protein, 9 aa, synthetic construct
  source 1..9: note = 2nd-Camp#84
AQRSIVKMP

SEQ ID NO: 116 - protein, 9 aa, synthetic construct
  source 1..9: note = 2nd-Camp#85
AQGKASYFP

SEQ ID NO: 117 - protein, 9 aa, synthetic construct
  source 1..9: note = 2nd-Camp#86
AQTASYRLR

SEQ ID NO: 118 - protein, 9 aa, synthetic construct
  source 1..9: note = 2nd-Camp#87
AQRSLPGPR

SEQ ID NO: 119 - protein, 9 aa, synthetic construct
  source 1..9: note = 2nd-Camp#88
AQIFTKGSR

SEQ ID NO: 120 - protein, 9 aa, synthetic construct
  source 1..9: note = 2nd-Camp#89
AQWPPATTP

SEQ ID NO: 121 - protein, 9 aa, synthetic construct
  source 1..9: note = 2nd-Camp#90
AQGNPMPRM

SEQ ID NO: 122 - protein, 9 aa, synthetic construct
  source 1..9: note = 2nd-Camp#91
AQWGQRRPG

SEQ ID NO: 123 - protein, 9 aa, synthetic construct
  source 1..9: note = 2nd-Camp#92
DGPMGVIRR

SEQ ID NO: 124 - protein, 9 aa, synthetic construct
  source 1..9: note = 2nd-Camp#93
AQEQPYRVR

SEQ ID NO: 125 - protein, 9 aa, synthetic construct
  source 1..9: note = 2nd-Camp#94
AQGLRRRMA

SEQ ID NO: 126 - protein, 9 aa, synthetic construct
  source 1..9: note = 2nd-Camp#95
AQRLEKRGS

SEQ ID NO: 127 - protein, 9 aa, synthetic construct
  source 1..9: note = 2nd-Camp#96
AQEVGRWRV

SEQ ID NO: 128 - protein, 9 aa, synthetic construct
  source 1..9: note = 2nd-Camp#97
AQSSKKMTI

SEQ ID NO: 129 - protein, 9 aa, synthetic construct
  source 1..9: note = 2nd-Camp#98
AQRSAPRGF

SEQ ID NO: 130 - protein, 9 aa, synthetic construct
  source 1..9: note = 2nd-Camp#99
AQKRHTEKA

SEQ ID NO: 131 - protein, 9 aa, synthetic construct
  source 1..9: note = 2nd-Camp#100
AQARAGARS

SEQ ID NO: 132 - protein, 9 aa, synthetic construct
  source 1..9: note = 2nd-Camp#101
AQWRSKWHV

SEQ ID NO: 133 - protein, 9 aa, synthetic construct
  source 1..9: note = 2nd-Camp#102
AQRLRLPAM

SEQ ID NO: 134 - protein, 9 aa, synthetic construct
  source 1..9: note = 2nd-Camp#103
DGPTIRRGR

SEQ ID NO: 135 - protein, 9 aa, synthetic construct
  source 1..9: note = 2nd-Camp#104
AQSRVRSGG

SEQ ID NO: 136 - protein, 9 aa, synthetic construct
  source 1..9: note = 2nd-Camp#105
AQSVLQFSK

SEQ ID NO: 137 - protein, 9 aa, synthetic construct
  source 1..9: note = 2nd-Camp#106
AQLRANRNS

SEQ ID NO: 138 - protein, 9 aa, synthetic construct
  source 1..9: note = 2nd-Camp#107
AQDRILGVS

SEQ ID NO: 139 - protein, 9 aa, synthetic construct
  source 1..9: note = 2nd-Camp#108
AQWRSLYLS

SEQ ID NO: 140 - protein, 9 aa, synthetic construct
  source 1..9: note = 2nd-Camp#109
DGPVMILPR

SEQ ID NO: 141 - protein, 9 aa, synthetic construct
  source 1..9: note = 2nd-Camp#110
AQQWTRLTP

SEQ ID NO: 142 - protein, 9 aa, synthetic construct
  source 1..9: note = 2nd-Camp#111
AQGWARRPP

SEQ ID NO: 143 - protein, 9 aa, synthetic construct
  source 1..9: note = 2nd-Camp#112
AQVRWVMRS

SEQ ID NO: 144 - protein, 9 aa, synthetic construct
  source 1..9: note = 2nd-Camp#113
AQTVRRPRG

SEQ ID NO: 145 - protein, 9 aa, synthetic construct
  source 1..9: note = 2nd-Camp#114
DGPVAYCIR

SEQ ID NO: 146 - protein, 9 aa, synthetic construct
  source 1..9: note = 2nd-Camp#115
DGPPPSLRR

SEQ ID NO: 147 - protein, 9 aa, synthetic construct
  source 1..9: note = 2nd-Camp#116
AQYMQLRSL

SEQ ID NO: 148 - protein, 9 aa, synthetic construct
  source 1..9: note = 2nd-Camp#117
AQRSGQWPL

SEQ ID NO: 149 - protein, 9 aa, synthetic construct
  source 1..9: note = 2nd-Camp#118
AQRLSVPRW

SEQ ID NO: 150 - protein, 9 aa, synthetic construct
  source 1..9: note = 2nd-Camp#119
AQYRPRGSA

SEQ ID NO: 151 - protein, 9 aa, synthetic construct
  source 1..9: note = 2nd-Camp#120
AQTPRKRLI

SEQ ID NO: 152 - protein, 9 aa, synthetic construct
  source 1..9: note = 2nd-Camp#121
AQRFPPGPS

SEQ ID NO: 153 - protein, 9 aa, synthetic construct
  source 1..9: note = 2nd-Camp#122
DGPGTRRTR

SEQ ID NO: 154 - protein, 9 aa, synthetic construct
  source 1..9: note = 2nd-Camp#123
DGPWYKYPR

SEQ ID NO: 155 - protein, 9 aa, synthetic construct
  source 1..9: note = 2nd-Camp#124
DGPSRRSPR

SEQ ID NO: 156 - protein, 9 aa, synthetic construct
  source 1..9: note = 2nd-Camp#125
AQRSHGRPG

SEQ ID NO: 157 - protein, 9 aa, synthetic construct
  source 1..9: note = 2nd-Camp#126
AQIMRQKPP

SEQ ID NO: 158 - protein, 9 aa, synthetic construct
  source 1..9: note = 2nd-Camp#127
AQLIGRKTV

SEQ ID NO: 159 - protein, 9 aa, synthetic construct
  source 1..9: note = 2nd-Camp#128
DGPKSRWVR

SEQ ID NO: 160 - protein, 9 aa, synthetic construct
  source 1..9: note = 2nd-Camp#129
AQWAPYRRW

SEQ ID NO: 161 - protein, 9 aa, synthetic construct
  source 1..9: note = 2nd-Camp#130
AQVSPGAAA

SEQ ID NO: 162 - protein, 9 aa, synthetic construct
  source 1..9: note = 2nd-Camp#131
AQQNSIRLV

SEQ ID NO: 163 - protein, 9 aa, synthetic construct
  source 1..9: note = 2nd-Camp#132
AQHKLPAWR

SEQ ID NO: 164 - protein, 9 aa, synthetic construct
  source 1..9: note = 2nd-Camp#133
DGPVRFMNR

SEQ ID NO: 165 - protein, 9 aa, synthetic construct
  source 1..9: note = 2nd-Camp#134
AQTASRYKP

SEQ ID NO: 166 - protein, 9 aa, synthetic construct
  source 1..9: note = 2nd-Camp#135
AQMSRQLRT

SEQ ID NO: 167 - protein, 9 aa, synthetic construct
  source 1..9: note = 2nd-Camp#136
AQPPRGRSF

SEQ ID NO: 168 - protein, 9 aa, synthetic construct
  source 1..9: note = 2nd-Camp#137
AQASSWRTS

SEQ ID NO: 169 - DNA, 27 bp, synthetic construct
  source 1..27: mol_type = other DNA; note = 164
gcccaacttcatgggttgcgggtgctt

SEQ ID NO: 170 - DNA, 27 bp, synthetic construct
  source 1..27: mol_type = other DNA; note = 165
gcccaacgtcaggggcttcggcttctg

SEQ ID NO: 171 - DNA, 27 bp, synthetic construct
  source 1..27: mol_type = other DNA; note = 166
gcccaaatgcggaggggtactacgaat

SEQ ID NO: 172 - DNA, 27 bp, synthetic construct
  source 1..27: mol_type = other DNA; note = 167
gcccaacttggtcgtagggtgatggtt

SEQ ID NO: 173 - DNA, 27 bp, synthetic construct
  source 1..27: mol_type = other DNA; note = 168
gcccaacggcttttgcgtattgagaag

SEQ ID NO: 174 - DNA, 27 bp, synthetic construct
  source 1..27: mol_type = other DNA; note = 169
gcccaatttgggaaggtttggaaggtg

SEQ ID NO: 175 - DNA, 27 bp, synthetic construct
  source 1..27: mol_type = other DNA; note = 170
gcccaagtgggtcggagggcgcttcct

SEQ ID NO: 176 - DNA, 27 bp, synthetic construct
  source 1..27: mol_type = other DNA; note = 171
gcccaacggtttggggggaggggtgct

SEQ ID NO: 177 - DNA, 27 bp, synthetic construct
  source 1..27: mol_type = other DNA; note = 172
gcccaacagccgcgtcttcgtgttggg

SEQ ID NO: 178 - DNA, 27 bp, synthetic construct
  source 1..27: mol_type = other DNA; note = 173
gcccaatctacgtctcggactcgggat

SEQ ID NO: 179 - DNA, 27 bp, synthetic construct
  source 1..27: mol_type = other DNA; note = 174
gcccaaaggagtcggggttcttatcct

SEQ ID NO: 180 - DNA, 27 bp, synthetic construct
  source 1..27: mol_type = other DNA; note = 175
gcccaagttgcttgggggcagcgtctg

SEQ ID NO: 181 - DNA, 27 bp, synthetic construct
  source 1..27: mol_type = other DNA; note = 176
gacggacctctgggggtgtatcggcgt

SEQ ID NO: 182 - DNA, 27 bp, synthetic construct
  source 1..27: mol_type = other DNA; note = 177
gcccaacgttctgctattgttcggatg

SEQ ID NO: 183 - DNA, 27 bp, synthetic construct
  source 1..27: mol_type = other DNA; note = 178
gacggacctcctacttggcgttcgcgt

SEQ ID NO: 184 - DNA, 27 bp, synthetic construct
  source 1..27: mol_type = other DNA; note = 179
gacggaccttcgctgaagtcgccgcgt

SEQ ID NO: 185 - DNA, 27 bp, synthetic construct
  source 1..27: mol_type = other DNA; note = 180
gcccaacggcttccgttgactcgtgct

SEQ ID NO: 186 - DNA, 27 bp, synthetic construct
  source 1..27: mol_type = other DNA; note = 181
gcccaagttggtcgttataaggtgagt

SEQ ID NO: 187 - DNA, 27 bp, synthetic construct
  source 1..27: mol_type = other DNA; note = 182
gcccaacgtcgtccgattcttctgggt

SEQ ID NO: 188 - DNA, 27 bp, synthetic construct
  source 1..27: mol_type = other DNA; note = 183
gacggacctaatccttatcggcttcgt

SEQ ID NO: 189 - DNA, 27 bp, synthetic construct
  source 1..27: mol_type = other DNA; note = 184
gacggacctgctcgtctgtctattcgt

SEQ ID NO: 190 - DNA, 27 bp, synthetic construct
  source 1..27: mol_type = other DNA; note = 185
gacggacctctgcggatgcctaatcgt

SEQ ID NO: 191 - DNA, 27 bp, synthetic construct
  source 1..27: mol_type = other DNA; note = 186
gcccaacggagtacgcttgtgaggagt

SEQ ID NO: 192 - DNA, 27 bp, synthetic construct
  source 1..27: mol_type = other DNA; note = 187
gacggacctggtccgggggtttcgcgt

SEQ ID NO: 193 - DNA, 27 bp, synthetic construct
  source 1..27: mol_type = other DNA; note = 188
gacggacctcgtaagtctacgtatcgt

SEQ ID NO: 194 - DNA, 27 bp, synthetic construct
  source 1..27: mol_type = other DNA; note = 189
gacggaccttgggttaattcgcctcgt

SEQ ID NO: 195 - DNA, 27 bp, synthetic construct
  source 1..27: mol_type = other DNA; note = 190
gacggacctaggccttatgctactcgt

SEQ ID NO: 196 - DNA, 27 bp, synthetic construct
  source 1..27: mol_type = other DNA; note = 191
gacggaccttgggtgaatacgccgcgt

SEQ ID NO: 197 - DNA, 27 bp, synthetic construct
  source 1..27: mol_type = other DNA; note = 192
gcccaatctcgggcgcggcctgctttg

SEQ ID NO: 198 - DNA, 27 bp, synthetic construct
  source 1..27: mol_type = other DNA; note = 193
gcccaaggtcggagtgagcagagtcct

SEQ ID NO: 199 - DNA, 27 bp, synthetic construct
  source 1..27: mol_type = other DNA; note = 194
gacggaccttattctcggactactcgt

SEQ ID NO: 200 - protein, 736 aa, synthetic construct
  source 1..736: note = aa sequence capctrl
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKAAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFNYEFENVPFHSSYAHSQSLDRLMNPLIDQYLYYLSKTINDSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADEVMITNEEEIKTTNPVATESYGQVAINHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 201 - DNA, 27 bp, synthetic construct
  source 1..27: mol_type = other DNA; note = X- 256-1
gcccaagtgatgaggctttcggctctt

SEQ ID NO: 202 - DNA, 27 bp, synthetic construct
  source 1..27: mol_type = other DNA; note = X-257-2
gcccaagtgaatggtctgcgtgtgctg

SEQ ID NO: 203 - DNA, 27 bp, synthetic construct
  source 1..27: mol_type = other DNA; note = X-258-3
gcccaacgtatgattaatgctgcgtgg

SEQ ID NO: 204 - DNA, 27 bp, synthetic construct
  source 1..27: mol_type = other DNA; note = X-259-4
gcccaacgggagggtctgcggcttctg

SEQ ID NO: 205 - DNA, 27 bp, synthetic construct
  source 1..27: mol_type = other DNA; note = X-260-5
gcccaacggcagaggcttgaggtgctt

SEQ ID NO: 206 - DNA, 27 bp, synthetic construct
  source 1..27: mol_type = other DNA; note = 2nd-Camp#6
gcccaacgtatgcgtctggagattctt

SEQ ID NO: 207 - DNA, 27 bp, synthetic construct
  source 1..27: mol_type = other DNA; note = 2nd-Camp#7
gcccaagctccgtcgtttcggtcttcg

SEQ ID NO: 208 - DNA, 27 bp, synthetic construct
  source 1..27: mol_type = other DNA; note = 2nd-Camp#8
gcccaaaaggttcatgtgcctcagtgg

SEQ ID NO: 209 - DNA, 27 bp, synthetic construct
  source 1..27: mol_type = other DNA; note = 2nd-Camp#9
gcccaacggggtgggctgagtgtgctt

SEQ ID NO: 210 - DNA, 27 bp, synthetic construct
  source 1..27: mol_type = other DNA; note = 2nd-Camp#10
gcccaaattcgtgggattagtgttatt

SEQ ID NO: 211 - DNA, 27 bp, synthetic construct
  source 1..27: mol_type = other DNA; note = 2nd-Camp#11
gcccaatttcgggggctttcttctctg

SEQ ID NO: 212 - DNA, 27 bp, synthetic construct
  source 1..27: mol_type = other DNA; note = 2nd-Camp#12
gcccaatattctcagacgtggcgggtt

SEQ ID NO: 213 - DNA, 27 bp, synthetic construct
  source 1..27: mol_type = other DNA; note = 2nd-Camp#13
gcccaagcggcttcgtggcggtcggcg

SEQ ID NO: 214 - DNA, 27 bp, synthetic construct
  source 1..27: mol_type = other DNA; note = 2nd-Camp#14
gcccaaatggaggggagttggcgtgtg

SEQ ID NO: 215 - DNA, 27 bp, synthetic construct
  source 1..27: mol_type = other DNA; note = 2nd-Camp#15
gcccaacgtcttgctcttcgtacgctt

SEQ ID NO: 216 - DNA, 27 bp, synthetic construct
  source 1..27: mol_type = other DNA; note = 2nd-Camp#16
gcccaacggatggggctgagtagtctt

SEQ ID NO: 217 - DNA, 27 bp, synthetic construct
  source 1..27: mol_type = other DNA; note = 2nd-Camp#17
gcccaatttcggcagttgtcggttctt

SEQ ID NO: 218 - DNA, 27 bp, synthetic construct
  source 1..27: mol_type = other DNA; note = 2nd-Camp#18
gcccaatttagggggtcgtggagtgtg

SEQ ID NO: 219 - DNA, 27 bp, synthetic construct
  source 1..27: mol_type = other DNA; note = 2nd-Camp#19
gcccaatttaagcctagttggtcggtg

SEQ ID NO: 220 - DNA, 27 bp, synthetic construct
  source 1..27: mol_type = other DNA; note = 2nd-Camp#20
gcccaaatgaagccgatttgggcggtg

SEQ ID NO: 221 - DNA, 27 bp, synthetic construct
  source 1..27: mol_type = other DNA; note = 2nd-Camp#21
gcccaactgatgcgtctgtctcttttg

SEQ ID NO: 222 - DNA, 27 bp, synthetic construct
  source 1..27: mol_type = other DNA; note = 2nd-Camp#22
gcccaaacgttgtatgttaataagtgg

SEQ ID NO: 223 - DNA, 27 bp, synthetic construct
  source 1..27: mol_type = other DNA; note = 2nd-Camp#23
gcccaacggaatgctcttcggagtctg

SEQ ID NO: 224 - DNA, 27 bp, synthetic construct
  source 1..27: mol_type = other DNA; note = 2nd-Camp#24
gcccaaagtgttacggttcctcggtgg

SEQ ID NO: 225 - DNA, 27 bp, synthetic construct
  source 1..27: mol_type = other DNA; note = 2nd-Camp#25
gcccaacgtctggcgaatccgcggtgg

SEQ ID NO: 226 - DNA, 27 bp, synthetic construct
  source 1..27: mol_type = other DNA; note = 2nd-Camp#26
gcccaaagggcgcgtgtggctgttgct

SEQ ID NO: 227 - DNA, 27 bp, synthetic construct
  source 1..27: mol_type = other DNA; note = X-261 #27
gcccaaattcggggtctgcgggttctg

SEQ ID NO: 228 - DNA, 27 bp, synthetic construct
  source 1..27: mol_type = other DNA; note = 2nd-Camp#28
gcccaactgactccgaggtggcatgtg

SEQ ID NO: 229 - DNA, 27 bp, synthetic construct
  source 1..27: mol_type = other DNA; note = 2nd-Camp#29
gcccaacatgttactatgtcgcggtgg

SEQ ID NO: 230 - DNA, 27 bp, synthetic construct
  source 1..27: mol_type = other DNA; note = 2nd-Camp#30
gcccaaatggggtcggtgtggcgggtg

SEQ ID NO: 231 - DNA, 27 bp, synthetic construct
  source 1..27: mol_type = other DNA; note = 2nd-Camp#31
gcccaagttctggggtatggtcattgg

SEQ ID NO: 232 - DNA, 27 bp, synthetic construct
  source 1..27: mol_type = other DNA; note = X-262 #32
gcccaatctatggggctgcgggttctg

SEQ ID NO: 233 - DNA, 27 bp, synthetic construct
  source 1..27: mol_type = other DNA; note = 2nd-Camp#33
gacggacctaatatggtttatagtcgt

SEQ ID NO: 234 - DNA, 27 bp, synthetic construct
  source 1..27: mol_type = other DNA; note = 2nd-Camp#34
gcccaaaatgcgttggcgtttgcgact

SEQ ID NO: 235 - DNA, 27 bp, synthetic construct
  source 1..27: mol_type = other DNA; note = 2nd-Camp#35
gcccaaaagcgtatgtcgtatcctgcg

SEQ ID NO: 236 - DNA, 27 bp, synthetic construct
  source 1..27: mol_type = other DNA; note = 2nd-Camp#36
gcccaatatagggctctgtctacgctg

SEQ ID NO: 237 - DNA, 27 bp, synthetic construct
  source 1..27: mol_type = other DNA; note = 2nd-Camp#37
gcccaaacggtgtatacggctcgttgg

SEQ ID NO: 238 - DNA, 27 bp, synthetic construct
  source 1..27: mol_type = other DNA; note = 2nd-Camp#38
gcccaatgggggcagaggtggcatgtt

SEQ ID NO: 239 - DNA, 27 bp, synthetic construct
  source 1..27: mol_type = other DNA; note = 2nd-Camp#39
gcccaaaggcatccggttgggttgggt

SEQ ID NO: 240 - DNA, 27 bp, synthetic construct
  source 1..27: mol_type = other DNA; note = 2nd-Camp#40
gcccaacgtcgttcgtctcctagtact

SEQ ID NO: 241 - DNA, 27 bp, synthetic construct
  source 1..27: mol_type = other DNA; note = 2nd-Camp#41
gcccaatggaatgcgcggtggcatgtg

SEQ ID NO: 242 - DNA, 27 bp, synthetic construct
  source 1..27: mol_type = other DNA; note = 2nd-Camp#42
gcccaattttcggcgcgttggagtgtg

SEQ ID NO: 243 - DNA, 27 bp, synthetic construct
  source 1..27: mol_type = other DNA; note = 2nd-Camp#43
gcccaagagaggggtcgttctagtagg

SEQ ID NO: 244 - DNA, 27 bp, synthetic construct
  source 1..27: mol_type = other DNA; note = 2nd-Camp#44
gcccaaaggctgccggcgcgtgttagt

SEQ ID NO: 245 - DNA, 27 bp, synthetic construct
  source 1..27: mol_type = other DNA; note = 2nd-Camp#45
gcccaaacgttgaggctgcgttctcgg

SEQ ID NO: 246 - DNA, 27 bp, synthetic construct
  source 1..27: mol_type = other DNA; note = 2nd-Camp#46
gcccaaccgcggtctaagatttctctt

SEQ ID NO: 247 - DNA, 27 bp, synthetic construct
  source 1..27: mol_type = other DNA; note = 2nd-Camp#47
gcccaatttccgagggttaggcggagg

SEQ ID NO: 248 - DNA, 27 bp, synthetic construct
  source 1..27: mol_type = other DNA; note = 2nd-Camp#48
gcccaacatcgtgggtctcgtgttccg

SEQ ID NO: 249 - DNA, 27 bp, synthetic construct
  source 1..27: mol_type = other DNA; note = 2nd-Camp#49
gcccaaatgatgacgcggaggcatgtt

SEQ ID NO: 250 - DNA, 27 bp, synthetic construct
  source 1..27: mol_type = other DNA; note = 2nd-Camp#50
gcccaacatccgctgcgtcgggtgtgg

SEQ ID NO: 251 - DNA, 27 bp, synthetic construct
  source 1..27: mol_type = other DNA; note = 2nd-Camp#51
gcccaagggtcgaagctgcttgggcgt

SEQ ID NO: 252 - DNA, 27 bp, synthetic construct
  source 1..27: mol_type = other DNA; note = 2nd-Camp#52
gacggaccttatttggggttgtttcgt

SEQ ID NO: 253 - DNA, 27 bp, synthetic construct
  source 1..27: mol_type = other DNA; note = 2nd-Camp#53
gcccaatcttatcctcatgcgaggggt

SEQ ID NO: 254 - DNA, 27 bp, synthetic construct
  source 1..27: mol_type = other DNA; note = 2nd-Camp#54
gcccaatggcatgagtttcattctccg

SEQ ID NO: 255 - DNA, 27 bp, synthetic construct
  source 1..27: mol_type = other DNA; note = 2nd-Camp#55
gcccaaaggcagaatcgtggtccgctt

SEQ ID NO: 256 - DNA, 27 bp, synthetic construct
  source 1..27: mol_type = other DNA; note = 2nd-Camp#56
gcccaatggtctaataagtggcatgtg

SEQ ID NO: 257 - DNA, 27 bp, synthetic construct
  source 1..27: mol_type = other DNA; note = 2nd-Camp#57
gcccaacgtggggcgattaattttcgt

SEQ ID NO: 258 - DNA, 27 bp, synthetic construct
  source 1..27: mol_type = other DNA; note = 2nd-Camp#58
gcccaaaggacgtattctgtgtctgcg

SEQ ID NO: 259 - DNA, 27 bp, synthetic construct
  source 1..27: mol_type = other DNA; note = 2nd-Camp#59
gcccaattgttgcatcggaatcgtagg

SEQ ID NO: 260 - DNA, 27 bp, synthetic construct
  source 1..27: mol_type = other DNA; note = 2nd-Camp#60
gcccaatgggcgtttcagggtcgtggg

SEQ ID NO: 261 - DNA, 27 bp, synthetic construct
  source 1..27: mol_type = other DNA; note = 2nd-Camp#61
gcccaacgggcgtctctgaggacgctg

SEQ ID NO: 262 - DNA, 27 bp, synthetic construct
  source 1..27: mol_type = other DNA; note = 2nd-Camp#62
gcccaacggtctctgcattcgcggacg

SEQ ID NO: 263 - DNA, 27 bp, synthetic construct
  source 1..27: mol_type = other DNA; note = 2nd-Camp#63
gcccaaccgaagcggccggttgctatt

SEQ ID NO: 264 - DNA, 27 bp, synthetic construct
  source 1..27: mol_type = other DNA; note = 2nd-Camp#64
gcccaacatacttttcggagtgtggct

SEQ ID NO: 265 - DNA, 27 bp, synthetic construct
  source 1..27: mol_type = other DNA; note = 2nd-Camp#65
gacggacctgttgttaggtctcggcgt

SEQ ID NO: 266 - DNA, 27 bp, synthetic construct
  source 1..27: mol_type = other DNA; note = X-263 #66
gcccaagatcgtgggttgagggtgttg

SEQ ID NO: 267 - DNA, 27 bp, synthetic construct
  source 1..27: mol_type = other DNA; note = 2nd-Camp#67
gacggacctcatttttctcggacgcgt

SEQ ID NO: 268 - DNA, 27 bp, synthetic construct
  source 1..27: mol_type = other DNA; note = 2nd-Camp#68
gcccaattgaggccggtgaagctgctg

SEQ ID NO: 269 - DNA, 27 bp, synthetic construct
  source 1..27: mol_type = other DNA; note = 2nd-Camp#69
gcccaatttcggcctgttcctagggcg

SEQ ID NO: 270 - DNA, 27 bp, synthetic construct
  source 1..27: mol_type = other DNA; note = 2nd-Camp#70
gcccaattgaatgtggttcgtcgtgtg

SEQ ID NO: 271 - DNA, 27 bp, synthetic construct
  source 1..27: mol_type = other DNA; note = 2nd-Camp#71
gcccaacgtattgctcgtcctcctttt

SEQ ID NO: 272 - DNA, 27 bp, synthetic construct
  source 1..27: mol_type = other DNA; note = 2nd-Camp#72
gcccaagcttctgctatgaagcgtcct

SEQ ID NO: 273 - DNA, 27 bp, synthetic construct
  source 1..27: mol_type = other DNA; note = 2nd-Camp#73
gcccaagttcgtcctagtgttaaggcg

SEQ ID NO: 274 - DNA, 27 bp, synthetic construct
  source 1..27: mol_type = other DNA; note = 2nd-Camp#74
gcccaagatcggaagcagcggccttcg

SEQ ID NO: 275 - DNA, 27 bp, synthetic construct
  source 1..27: mol_type = other DNA; note = 2nd-Camp#75
gcccaaaagaatgttcctaaggcgtct

SEQ ID NO: 276 - DNA, 27 bp, synthetic construct
  source 1..27: mol_type = other DNA; note = 2nd-Camp#76
gcccaaggtctgtcgcgttgggctccg

SEQ ID NO: 277 - DNA, 27 bp, synthetic construct
  source 1..27: mol_type = other DNA; note = 2nd-Camp#77
gcccaatttaagcataagggtcttatt

SEQ ID NO: 278 - DNA, 27 bp, synthetic construct
  source 1..27: mol_type = other DNA; note = 2nd-Camp#78
gcccaacggttggcgttgccgaggtct

SEQ ID NO: 279 - DNA, 27 bp, synthetic construct
  source 1..27: mol_type = other DNA; note = 2nd-Camp#79
gcccaacgtccgccgaggcgttcgggt

SEQ ID NO: 280 - DNA, 27 bp, synthetic construct
  source 1..27: mol_type = other DNA; note = 2nd-Camp#80
gcccaaacgcgggggccgtggagggtg

SEQ ID NO: 281 - DNA, 27 bp, synthetic construct
  source 1..27: mol_type = other DNA; note = 2nd-Camp#81
gacggacctacgattcggtatcttcgt

SEQ ID NO: 282 - DNA, 27 bp, synthetic construct
  source 1..27: mol_type = other DNA; note = 2nd-Camp#82
gcccaacctcggaggactcgtgttgcg

SEQ ID NO: 283 - DNA, 27 bp, synthetic construct
  source 1..27: mol_type = other DNA; note = 2nd-Camp#83
gcccaaagtagggcgagtcgtgtttcg

SEQ ID NO: 284 - DNA, 27 bp, synthetic construct
  source 1..27: mol_type = other DNA; note = 2nd-Camp#84
gcccaacggtcgattgttaagatgcct

SEQ ID NO: 285 - DNA, 27 bp, synthetic construct
  source 1..27: mol_type = other DNA; note = 2nd-Camp#85
gcccaagggaaggcttcgtattttcct

SEQ ID NO: 286 - DNA, 27 bp, synthetic construct
  source 1..27: mol_type = other DNA; note = 2nd-Camp#86
gcccaaactgcgtcttatcgtctgcgt

SEQ ID NO: 287 - DNA, 27 bp, synthetic construct
  source 1..27: mol_type = other DNA; note = 2nd-Camp#87
gcccaacggtctcttccgggtcctcgg

SEQ ID NO: 288 - DNA, 27 bp, synthetic construct
  source 1..27: mol_type = other DNA; note = 2nd-Camp#88
gcccaaatttttacgaaggggagtcgt

SEQ ID NO: 289 - DNA, 27 bp, synthetic construct
  source 1..27: mol_type = other DNA; note = 2nd-Camp#89
gcccaatggccgcctgcgactactccg

SEQ ID NO: 290 - DNA, 27 bp, synthetic construct
  source 1..27: mol_type = other DNA; note = 2nd-Camp#90
gcccaaggtaatccgatgccgaggatg

SEQ ID NO: 291 - DNA, 27 bp, synthetic construct
  source 1..27: mol_type = other DNA; note = 2nd-Camp#91
gcccaatggggtcagaggcggcctggt

SEQ ID NO: 292 - DNA, 27 bp, synthetic construct
  source 1..27: mol_type = other DNA; note = 2nd-Camp#92
gacggacctatgggggttattcggcgt

SEQ ID NO: 293 - DNA, 27 bp, synthetic construct
  source 1..27: mol_type = other DNA; note = 2nd-Camp#93
gcccaagagcagccttatagggttcgg

SEQ ID NO: 294 - DNA, 27 bp, synthetic construct
  source 1..27: mol_type = other DNA; note = 2nd-Camp#94
gcccaaggtcttcggcggcgtatggct

SEQ ID NO: 295 - DNA, 27 bp, synthetic construct
  source 1..27: mol_type = other DNA; note = 2nd-Camp#95
gcccaacggcttgagaagcgggggtcg

SEQ ID NO: 296 - DNA, 27 bp, synthetic construct
  source 1..27: mol_type = other DNA; note = 2nd-Camp#96
gcccaagaggtggggcgttggcgggtg

SEQ ID NO: 297 - DNA, 27 bp, synthetic construct
  source 1..27: mol_type = other DNA; note = 2nd-Camp#97
gcccaatctagtaagaagatgactatt

SEQ ID NO: 298 - DNA, 27 bp, synthetic construct
  source 1..27: mol_type = other DNA; note = 2nd-Camp#98
gcccaaaggtcggctcctcgtgggttt

SEQ ID NO: 299 - DNA, 27 bp, synthetic construct
  source 1..27: mol_type = other DNA; note = 2nd-Camp#99
gcccaaaagcgtcatacggagaaggcg

SEQ ID NO: 300 - DNA, 27 bp, synthetic construct
  source 1..27: mol_type = other DNA; note = 2nd-Camp#100
gcccaagcgcgggcgggtgcgcggtcg

SEQ ID NO: 301 - skipped

SEQ ID NO: 302 - protein, 9 aa, synthetic construct
  source 1..9: note = consensus claim 8
  VARIANT 3..4: note = X may be any amino acid.
  VARIANT 6..8: note = X may be any amino acid.
AQXXGXXXL

SEQ ID NO: 303 - protein, 9 aa, synthetic construct
  source 1..9: note = consensus 2 claim 8
  VARIANT 7: note = X may be any amino acid.
DGPWVNXPR

SEQ ID NO: 304 - protein, 9 aa, synthetic construct
  source 1..9: note = consensus pg 9
  VARIANT 3..4: note = X may be any amino acid.
  VARIANT 8: note = X may be any amino acid.
AQXXGLRXL

SEQ ID NO: 305 - protein, 7 aa, synthetic construct
  source 1..7: note = consensus examples
  VARIANT 1..2: note = X may be any amino acid.
  VARIANT 6: note = X may be any amino acid.
XXGLRXL

SEQ ID NO: 306 - protein, 736 aa, synthetic construct; identical to SEQ ID NO: 200
  source 1..736: note = capctrl

SEQ ID NO: 307 - protein, 736 aa, synthetic construct; identical to SEQ ID NO: 200
  source 1..736: note = capctrl-wt

SEQ ID NO: 308 - protein, 743 aa, synthetic construct
  source 1..743: note = consensus sequence AAV9 wt
  VARIANT 589..595: note = X may be any amino acid.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQXXXXXXXAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 309 - protein, 736 aa, synthetic construct
  source 1..736: note = capctrl 453D
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 310 - protein, 743 aa, synthetic construct
  source 1..743: note = consensus capctrl
  VARIANT 589..595: note = X may be any amino acid.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKAAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFNYEFENVPFHSSYAHSQSLDRLMNPLIDQYLYYLSKTINDSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADEVMITNEEEIKTTNPVATESYGQVAINHQSAQXXXXXXXAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 311 - protein, 743 aa, synthetic construct; identical to SEQ ID NO: 308
  source 1..743: note = consensus AAV9 453D
  VARIANT 589..595: note = X may be any amino acid.

(19) United States
(12) Patent Application Publication
(10) Pub. No.: US 2026/0125704 A1
(43) Pub. Date: May 7, 2026

(54) rAAV capsids

(71) Applicant: uniQure biopharma B.V., Amsterdam (NL)

(72) Inventors: - Anggakusuma, Amsterdam (NL); Elena Maria Santidrian Yebra-Pimentel, Amsterdam (NL)

(73) Assignee: uniQure biopharma B.V., Amsterdam (NL)

(21) Appl. No.: 19/307,085

(22) Filed: Aug. 22, 2025

(30) Foreign Application Priority Data
Aug. 22, 2024 (EP) 24195857.8

(51) Int. Cl. C12N 15/86; A61K 48/00

(52) U.S. Cl. CPC C12N 15/86; A61K 48/00; C12N 2750/14143; C12N 2750/14151

(57) ABSTRACT

The present invention relates to the fields of medicine, molecular biology, and gene therapy. In particular, the invention relates to novel recombinant adeno-associated virus capsids, uses thereof and methods of manufacturing.

### FIELD OF THE INVENTION

The present invention relates to the fields of medicine, molecular biology, and gene therapy. In particular, the invention relates to novel recombinant adeno-associated virus capsids, uses thereof and methods of manufacturing the novel recombinant adeno-associated virus capsids.

### REFERENCE TO AN ELECTRONIC SEQUENCE LISTING

The contents of the electronic sequence listing (sequencelisting.txt; Size: 334.460 bytes; and Date of Creation: Aug. 22, 2025) is herein incorporated by reference in its entirety.

### BACKGROUND OF THE INVENTION

Recombinant adeno-associated virus (rAAV) has emerged as a preferred platform for gene therapy. Yet, the development of novel rAAV-based therapeutic strategies has faced several hurdles. Specifically, developing novel rAAV capsids that can transduce cells in the central nervous system (CNS) upon systemic administration is one of the challenges in the field of rAAV-based gene therapy of CNS diseases. The inefficiency of blood-brain barrier (BBB) crossing limits the CNS application of first generation rAAV-based capsids due to the narrow therapeutic window when administered systemically. Traditional methods, such as intraparenchymal injection and intrathecal infusion, are invasive and may provide limited coverage. To address these challenges, research is being conducted on capsid engineering to develop novel rAAV capsids that can efficiently cross the BBB and achieve widespread CNS transduction via less invasive routes of administration, such as systemic delivery.

A major milestone in this field is the engineering of rAAV capsids via peptide insertion, which has led to the development of e.g. PHP.B rAAV capsid variants—the first to demonstrate efficacy in transducing the brain of C57BL/6 mice following systemic injection (Deverman et al., 2016, Nat Biotechnol., 34(2): 204-209). However, these engineered rAAV capsid variants did not perform consistently across different mouse strains or in non-human primates (NHPs). Consequently, various research groups have employed different rAAV capsid engineering platforms to create engineered capsids with peptide insertions capable of crossing the BBB beyond rodent models and efficiently transducing the brain (Lopez-Gordo et al., Viruses 2024, 16(3), 442).

There remains, however, a need for further improved rAAV capsids that can cross the BBB and efficiently transduce cells in the CNS upon systemic administration. It is an object of the invention to provide engineered rAAV capsids with the capacity to cross the BBB.

### SUMMARY OF THE INVENTION

In a first aspect, there is provided a recombinant adeno-associated virus (rAAV) capsid comprising at least one binding moiety, wherein the at least one binding moiety confers to the rAAV capsid a phenotype of increased interaction with carbonic anhydrase 4 (CA4) as compared to a control rAAV capsid not comprising the binding moiety.

In one embodiment, the rAAV capsid presents at least one of the following phenotypes upon systemic administration: i) a phenotype of increased blood-brain barrier (BBB) crossing as compared to a control rAAV capsid not comprising the binding moiety; and/or ii) a phenotype of increased transduction of cells in the central nervous system (CNS) as compared to a control rAAV capsid not comprising the binding moiety.

In one embodiment, the binding moiety comprises an amino acid insert between two consecutive amino acids present within amino acids 580-595, preferably amino acids 585-590, more preferably amino acids 588 and 589 of an AAV9 capsid protein, or in an analogous position of a capsid protein of an AAV capsid serotype selected from the group comprising: AAV1, AAV2, AAV3A, AAV3B, AAV4, AAV5, AAV6, AAV7, AAV8, AAV10, AAV11, AAV12, AAV13 and AAVrh10, or modified versions thereof, preferably AAV6, or modified versions thereof.

In one embodiment, the binding moiety comprises at least 4, 5, 6, or 7 contiguous amino acids of an amino acid sequence X₁X₂X₃X₄X₅X₆X₇ (SEQ ID NO: 301), wherein X₁ is selected from: L, R, V, I, S, D, A, K and P; X₂ is selected from: H, Q, A, N, E, R, M, G, P, V and W; X₃ is selected from: G, W, R, S, H, I and V; X₄ is selected from: L, I, G, F, V and N; X₅ is selected from: R, Q, S, E, P, A and T; X₆ is selected from: V, L, R, A, I, S, Q and P, and; X₇ is selected from: L, I, S, W and R; optionally wherein the amino acid at position 587 is mutated to D, and/or the amino acid at position 588 is mutated to G.

In one embodiment, the binding moiety does not compete with brinzolamide for interaction with CA4.

In one embodiment, the rAAV capsid comprises an expression cassette flanked by at least one AAV inverted terminal repeat (ITR), wherein the expression cassette comprises a nucleic acid molecule encoding at least one gene product.

In a second aspect, there is provided a polynucleotide encoding an rAAV capsid as described herein.

In a third aspect, there is provided a host cell comprising a polynucleotide for expression of the rAAV capsid as described herein.

In a fourth aspect, there is provided a composition comprising the rAAV capsid as described herein, preferably wherein the composition is a pharmaceutical composition further comprising at least one pharmaceutically acceptable carrier.

In a fifth aspect there is provided an rAAV capsid as described herein, or a composition as described herein for use as a medicament, preferably for use in the treatment of a condition of the central nervous system.

In a sixth aspect, there is provided an rAAV capsid as described herein, or a composition as described herein, for use as a medicament in gene therapy.

In one embodiment, there is provided a method of gene therapy comprising the step of administering an effective amount of an rAAV capsid as described herein, or a composition comprising the rAAV capsid as described herein, to a subject in need of gene therapy.

In a seventh aspect, there is provided a method of manufacturing an rAAV capsid as described herein, the method comprising the steps of: I) culturing a host cell comprising: i) an expression vector for expression of an rAAV capsid as described herein, and; ii) an expression vector encoding an expression cassette flanked by at least one AAV inverted terminal repeat (ITR), comprising a nucleic acid molecule encoding at least one gene product; said host cell preferably further comprising a nucleotide sequence encoding a parvoviral replication (Rep) protein under conditions such that the rAAV capsid is produced; and, II) recovery of the rAAV capsid, preferably wherein recovery of the rAAV capsid comprises at least one of: affinity-purification of the rAAV capsid using an immobilized anti-AAV antibody, preferably a single chain camelid antibody or a fragment thereof, or filtration using a filter having a nominal pore size of 30-70 nm.

### DESCRIPTION OF THE INVENTION

The present invention surprisingly provides multiple rAAV capsid variants that interact with human Carbonic Anhydrase 4 (hCA4), enabling BBB crossing of the capsids.

Recombinant Adeno-Associated Virus (rAAV) Capsids

In a first aspect, there is provided a recombinant adeno-associated virus (rAAV) capsid comprising at least one binding moiety, wherein the at least one binding moiety confers to the rAAV capsid a phenotype of increased interaction with carbonic anhydrase 4 (CA4) as compared to a control rAAV capsid not comprising the binding moiety.

The term “capsid” as used herein may interchangeably be denominated by the terms “viral vector”, “virion”, “vector virion”, “capsid variant” and/or “particle”. The term “capsid” as used herein may refer to a collection of capsid proteins that together assemble into an AAV or rAAV capsid, and may as well refer to the combination of an AAV or rAAV capsid and any cargo that is contained within the capsid. Exemplary cargo includes, but is not limited to an expression vector.

The term “recombinant” in relation to the capsids as described herein is used to indicate that the recombinant AAV capsids comprise mutations that were intentionally introduced, distinguishing them from wild type AAV capsids having the same serotype. This means that the amino acid sequence of the rAAV capsid as described herein is non-naturally occurring. As used herein, the term “non-naturally occurring” when used in reference to an rAAV capsid means that the rAAV capsid comprises at least one mutation that is not normally found in naturally occurring AAV capsids, including wild-type AAV capsids.

The term “binding moiety” as used herein refers to a peptide, a polypeptide or oligopeptide having the capacity to specifically bind to a certain tertiary or quaternary protein structure or epitope. In some embodiments, a binding moiety as referred to herein may also consist of or comprise carbohydrate chains having the capacity to specifically bind to a certain tertiary or quaternary protein structure or epitope.

The terms “interaction” and “interacting” as used herein may be used interchangeably with terms including, but not limited to: “association, associating, connection, connecting, binding, engaging”. The term interaction as used herein indicates that two entities specifically bind to each other, but that the binding is of a transient nature, preferably wherein the binding event is of a transient nature. This transient nature of the binding event allows the rAAV capsids as described herein to cross the BBB, followed by release into the CNS.

The terms “control rAAV capsid not comprising the binding moiety”, “control rAAV capsid not capable of interacting with CA4” or “control rAAV capsid” as used herein may be used interchangeably and refer to any rAAV capsid of any serotype. The control rAAV capsid should not comprise a binding moiety that interacts with CA4, and the rAAV serotype should not intrinsically interact with CA4 to a higher degree as compared to wild type AAV capsids of the different serotypes, including, but not limited to: AAV1, AAV2, AAV3A, AAV3B, AAV4, AAV5, AAV6, AAV7, AAV8, AAV9, AAV10, AAV11, AAV12, AAV13 and AAVrh10, preferably AAV9. Both rAAV and AAV capsids may be used as control capsids. The degree to which a control capsid interacts with CA4, including murine, macaque and human CA4, may be determined using methods as described herein.

Where herein reference is made to “Carbonic Anhydrase 4 (CA4)”, it is understood to refer to human Carbonic Anhydrase 4 (hCA4). Therefore, in one embodiment there is provided a recombinant adeno-associated virus (rAAV) capsid comprising a binding moiety, wherein the binding moiety confers to the rAAV capsid a phenotype of increased interaction with human carbonic anhydrase 4 (CA4) as compared to a control capsid not comprising the binding moiety.

The term “Carbonic Anhydrase 4 (CA4)” may also be referred to herein as “Carbonate dehydratase IV” and/or “Carbonic anhydrase IV (CA-IV)”. Human CA4 is herein understood to comprise, consist of, or to be comprised within the amino acid denominated by UniProt® Database Accession No. P22748 and the nucleotide sequence denominated by HGNC ID: 1375 (CA4). CA4 is a glycosylphosphatidylinositol (GPI)-anchored membrane protein expressed on the surface of brain microvascular endothelial cells (BMEC), which are important components of the microvasculature forming the BBB. Without being bound by theory, it is believed that capsids as described herein when present in the blood can associate with CA4 expressed on BMEC via binding moieties as described herein. Upon association with CA4 the capsids can leave the blood circulation and enter the cerebrospinal fluid (CSF) through a transcellular or possibly paracellular mechanism. Upon dissociation from CA4, the capsids of the invention are released into the CSF, enabling the capsids to transduce target cells of the CNS, e.g. astrocytes, microglia and neurons.

In one embodiment, the phenotype of increased interaction with CA4 is determined by incubating an rAAV capsid as described herein comprising a reporter gene, with CA4-expressing cells, preferably CA4-expressing Human Embryonic Kidney (HEK) 293T cells, and determining the number of cells that are positive for the reporter gene, and preferably comparing the number of cells that are positive for the reporter gene upon incubation with an rAAV capsid as described herein with the number of cells that are positive for the reporter gene when incubated with a control rAAV capsid not capable of interacting with CA4, preferably under otherwise identical conditions.

In one embodiment, the number of cells that are positive for the reporter gene is at least 2, 3, 4, 5, 10, 15, 20, 40, 50, 60, 75 or 100 fold higher when incubated with an rAAV capsid as described herein as compared to a control rAAV capsid not capable of interacting with CA4.

Exemplary methods for determining interaction of rAAV capsids as described herein with CA4 include, but are not limited to: polymerase-chain-reaction-based methods, protein expression analysis, deep sequencing, electron microscopy and fluorescence microscopy.

In one embodiment, the rAAV capsid presents at least one of the following phenotypes upon systemic administration: i) a phenotype of increased blood-brain barrier (BBB) crossing as compared to a control rAAV capsid not comprising the binding moiety; and/or ii) a phenotype of increased transduction of cells in the central nervous system (CNS) as compared to a control rAAV capsid not comprising the binding moiety.

Where herein reference is made to “blood-brain barrier (BBB) crossing” it is intended to refer to the crossing of the BBB from the lumen of the blood vessel towards the CNS.

In one embodiment, the phenotype of increased BBB crossing is determined in vivo, by administering an rAAV capsid comprising a binding moiety as described herein to a non-human animal, preferably a mammal, and determining whether rAAV capsids reach the CNS, and preferably comparing the number of rAAV capsids that are present in the CNS to the number of control rAAV capsids that are present in the CNS, preferably under otherwise identical conditions. In a preferred embodiment, the phenotype of BBB crossing is determined upon systemic administration of the rAAV capsids as described herein to a non-human animal.

In one embodiment, the phenotype of BBB crossing is determined 1, 4, 8, 24 or 48 hours post-administration. In one embodiment, the number of capsids that is present in the CNS is at least 2, 3, 4, 5, 10, 15, 20, 40, 50, 60, 75 or 100 fold higher upon administration of an rAAV capsid as described herein, as compared to a control rAAV capsid. In one embodiment, the non-human animal is a mouse or other laboratory research animal, such as, but not limited to, a rabbit, a rat, a dog or a non-human primate.

Exemplary methods for determining the presence of rAAV capsids in the CNS include, but are not limited to: polymerase-chain-reaction-based methods, protein expression analysis, deep sequencing, electron microscopy and fluorescence.

In one embodiment, the phenotype of increased BBB crossing is determined using an in vitro method as provided in the examples.

In one embodiment, the phenotype of increased transduction of cells in the CNS is determined by administering an rAAV capsid as described herein to a non-human animal and determining the transduction levels of cells in the CNS, and preferably comparing the transduction levels obtained using an rAAV capsid as described herein with the transduction levels obtained using a control rAAV capsid, preferably under otherwise identical conditions. In a preferred embodiment the phenotype of increased transduction of cells in the CNS is determined upon systemic administration of the rAAV capsids as described herein to a non-human animal.

In one embodiment, the phenotype of increased transduction of cells in the CNS is determined 2, 3, 4, 5 or 6 weeks post-administration. In one embodiment, the transduction level of cells in the CNS is at least 2, 3, 4, 5, 10, 15, 20, 40, 50, 60, 75 or 100 fold higher upon administration of an rAAV capsid as described herein, as compared to a control rAAV capsid. In one embodiment, the non-human animal is a mouse or other research animal, such as, but not limited to, a rabbit, a rat, a dog or a non-human primate.

Exemplary methods for determining transduction of cells in the CNS include, but are not limited to: polymerase-chain-reaction-based methods, protein expression analysis, deep sequencing, electron microscopy and fluorescence microscopy.

In one embodiment, the phenotype of increased transduction of cells in the CNS is determined using an in vitro method as provided in the examples.

In one embodiment, at least part of the binding moiety comprised in the rAAV capsid is inserted between two consecutive amino acids of a capsid protein.

The term “at least part of” as used herein means to indicate that at least 1 amino acid, but possibly more, e.g. 2, 3, 4, 5, 6, 7 or more amino acids of the binding moiety are additional to the amino acid sequence of a wild type AAV capsid protein. This means that the amino acid sequence of a capsid protein constituting the rAAV capsid as described herein is at least 1 amino acid, possibly more, e.g. 2, 3, 4, 5, 6, 7 or more amino acids longer as compared to the amino acid sequence of a corresponding wild type AAV capsid protein.

The term “inserted between” as used herein refers to an insertion or addition of additional amino acids into the polypeptide constituting an rAAV capsid protein.

The term “consecutive amino acids” refers to the situation wherein for example a first amino acid of a polypeptide is followed by a second amino acid, or a fifth amino acid is followed by a sixth amino acid. In one embodiment, at least part of the binding moiety as described herein is added between two amino acids that are adjacent in at least one polypeptide that is part of an AAV capsid protein.

AAV capsids are assembled from viral proteins VP1, VP2 and VP3. In one embodiment, the binding moiety comprised in the rAAV capsid is inserted between two consecutive amino acids of at least one of VP1, VP2 and VP3. In a preferred embodiment, the binding moiety is inserted between two consecutive amino acids of VP1, VP2 and VP3. Together, multiple VP1, VP2 and VP3 monomers assemble into an AAV capsid.

In one embodiment, the rAAV capsid as described herein comprises at least two binding moieties, wherein a first binding moiety is present in a first viral protein, and a second binding moiety is present in a second viral protein, wherein the first and second viral protein are not the same.

In another embodiment, the rAAV capsid as described herein comprises at least two binding moieties, wherein a first binding moiety is present in a first viral protein, and a second binding moiety is present in the same viral protein.

In a preferred embodiment, the first binding moiety interacts with CA4, and the second binding moiety interacts with a different protein.

In a preferred embodiment, the first binding moiety interacts with CA4, and the second binding moiety interacts with a protein that is expressed on the surface of human brain microvascular endothelial cells (BMEC).

Exemplary proteins that are expressed on the surface of human brain microvascular endothelial cells (BMEC) include, but are not limited to: Transferrin Receptor 1 (TfR1/CD71, UniProt® Database Accession No. P02786), Cell cycle control protein 50A (UniProt® Database Accession No. Q9NV96), alkaline phosphatase—tissue-nonspecific isozyme (TNS-ALP, UniProt® Database Accession No. P05186), Low-density lipoprotein receptor-related protein 6 (LRP-6, UniProt® Database Accession No. 075581), and CD59 glycoprotein (CD59, UniProt® Database Accession No. P13987).

In a preferred embodiment, the at least one binding moiety is external to the rAAV capsid. The terms “external to the rAAV capsid” and “on the surface of the rAAV capsid” as used herein may be used interchangeably and refer to the amino acids forming secondary and tertiary structures facing outside of the rAAV capsid, as opposed to the amino acids forming secondary and tertiary structures facing the inside of the rAAV capsid. It may thus be interpreted as such, that the amino acids that are part of the binding moiety are part of a secondary and tertiary structure of the rAAV capsid facing outside of the rAAV capsid.

In one embodiment the binding moiety is comprised in a variable region (VR) of a surface loop of the rAAV capsid, preferably in at least one of the VR-I, VR-II, VR-III, VR-IV, VR-V, VR-VI, VR-VII, VR-VIII and/or VR-IX, preferably in the VR-IV and/or VR-VIII, more preferably in the VR-VIII.

AAV viral proteins have nine variable regions (VRs), which are oriented external to the AAV capsid. Individually, the variable regions shape the exterior of the AAV capsid. In a preferred embodiment, the binding moiety is comprised in the VR-VIII.

In one embodiment, the binding moiety comprised in the rAAV capsid comprises an amino acid insert between two consecutive amino acids present within amino acids 580-595, preferably amino acids 585-590, more preferably amino acids 588 and 589 of an AAV9 capsid protein, or in an analogous position of a capsid protein of an AAV capsid serotype selected from the group comprising: AAV1, AAV2, AAV3A, AAV3B, AAV4, AAV5, AAV6, AAV7, AAV8, AAV10, AAV11, AAV12, AAV13 and AAVrh10, or modified versions thereof, preferably AAV6, or modified versions thereof.

The skilled person will be aware that where herein reference is made to numbered amino acids, the amino acid numbered 1 is the amino-terminal amino acid of the capsid protein, from there, the amino acids count up or upwards sequentially and monotonically until the carboxy-terminal amino acid of the capsid protein. In one embodiment, the numbered amino acids referred to herein do not include the amino acids that constitute the insert itself, but refer to amino acids that were present in the (recombinant) AAV capsid protein prior to addition of the insert.

In one embodiment, the sequences coding for the VP1, VP2, and VP3 capsid proteins for use in the context of the present invention may be taken from any of the known 42 serotypes, more preferably from AAV1, AAV2, AAV3, AAV4, AAV5, AAV6, AAV7, AAV8. AAV9, AAV10, AAV11, AAV12, AAV13, AAVrh10, or AAVrh74, or from newly developed rAAV capsids obtained by e.g. capsid shuffling techniques.

In a further embodiment, modified AAV capsids, or “modified versions” are used. Modified AAV capsids have at least 70%, at least 75%, at least 80%, at least 85%, at least 90%, at least 95%, or more nucleotide and/or amino acid sequence identity, e.g., a sequence having 75-99% nucleotide sequence identity, to an AAV1, AAV2, AAV3, AAV4, AAV5, AAV6, AAV7, AAV8, AAV9, AAV10, AAV11, AAV12, AAV13, AAVrh10, or AAVrh74 capsid.

Where herein reference is made to “an analogous position of a capsid protein”, it is intended to refer to a position in a capsid protein of a serotype other than AAV9, that corresponds to a similar position as the position indicated in the AAV9 capsid protein. Such similarity may occur for instance due to a similar position of amino acids within a certain VR region. Analogous positions of a capsid protein may be determined using software, including protein or sequence-BLAST.

Without being bound to theory, AAV-serotypes that have similar capsid protein amino acid sequences, and comprise at least one binding moiety as described herein in analogous positions are expected to have similar functionality. Exemplary AAV serotypes with similar capsid protein amino acid sequences include, but are not limited to AAV9 and AAV6.

In one embodiment, the amino acid insert comprised in the binding moiety consists of 2, 3, 4, 5, 6, 7, 8, 9, 10, 11, 12, 13, 14, 15 or more amino acids, preferably 5-9 amino acids, more preferably 7 amino acids.

In one embodiment, the rAAV capsid comprises a binding moiety comprising at least 4, 5, 6, or 7 contiguous amino acids of an amino acid sequence X₁X₂X₃X₄X₅X₆X₇ (SEQ ID NO: 301), wherein: X₁ is selected from: L, R, V, I, S, D, A, K and P; X₂ is selected from: H, Q, A, N, E, R, M, G, P, V and W; X₃ is selected from: G, W, R, S, H, I and V; X₄ is selected from: L, I, G, F, V and N; X₅ is selected from: R, Q, S, E, P, A and T; X₆ is selected from: V, L, R, A, I, S, Q and P, and; X₇ is selected from: L, I, S, W and R; optionally wherein the amino acid at position 587 is mutated to D, and/or the amino acid at position 588 is mutated to G. Preferably wherein X₁ is inserted after amino acid 588 and X₇ is followed by amino acid 589 of the rAAV capsid protein. In a preferred embodiment, X₁ is inserted directly following amino acid 588 and X₇ is followed by amino acid 589 of an AAV9 capsid protein, or in an analogous position of a capsid protein of an AAV capsid serotype selected from the group comprising: AAV1, AAV2, AAV3A, AAV3B, AAV4, AAV5, AAV6, AAV7, AAV8, AAV10, AAV11, AAV12, AAV13 and AAVrh10, or modified versions thereof, preferably AAV6 or modified versions thereof.

In one embodiment, the rAAV capsid protein comprises or consists of SEQ ID NO: 306, wherein X₁ is inserted after amino acid 588 and X₇ is followed by amino acid 589.

In a preferred embodiment, the rAAV capsid protein comprises or consists of SEQ ID NO:306, 307 or 309, wherein X₁ is inserted after amino acid 588 and X₇ is followed by amino acid 589.

In one embodiment, the rAAV capsid protein comprises or consists of SEQ ID NO: 306, wherein X₁ is inserted after amino acid 588 and X₈ is followed by amino acid 589.

In a preferred embodiment, the rAAV capsid protein comprises or consists of SEQ ID NO:306, 307 or 309, wherein X₁ is inserted after amino acid 588 and X₈ is followed by amino acid 589.

In a preferred embodiment, the rAAV capsid protein comprises or consists of SEQ ID NO: 310, or a modified version thereof. In one embodiment, the modified AAV capsid protein has at least 70%, or at least 75%, or at least 80%, or at least 85%, preferably at least 90%, more preferably at least 95%, or more amino acid sequence identity with SEQ ID NO: 310.

In a preferred embodiment, the rAAV capsid protein comprises or consists of SEQ ID NO: 310, 308 or 311, or a modified version thereof. In one embodiment, the modified AAV capsid protein has at least 70%, or at least 75%, or at least 80%, or at least 85%, preferably at least 90%, more preferably at least 95%, or more amino acid sequence identity with SEQ ID NO: 310, 308 or 311.

In a preferred embodiment, the rAAV capsid comprises a binding moiety comprising a consensus sequence AQX₁X₂GX₃X₄X₅L (SEQ ID NO: 302), wherein X₁ is selected from: L, R, V, I, S and D; X₂ is selected from: H, Q, N, E, R and M; X₃ is selected from: L and I; X₄ is selected from: R and Q, and; X₅ is selected from: V, L and R.

In a further embodiment, the rAAV capsid comprises a binding moiety comprising a consensus sequence DGPWVNXPR (SEQ ID NO: 303), wherein X is selected from: S, and T.

In a preferred embodiment, the rAAV capsid comprises a binding moiety comprising a consensus sequence AQX₁X₂GLR X₃L (SEQ ID NO: 304). Preferably wherein X₁ is selected from S, L, V, D, I or R; X₂ is selected from M, H, N, R, G, Q or E; and X₃ is selected from V and L.

In a further embodiment, the rAAV capsid comprises a binding moiety, wherein the binding moiety comprises or consists of an amino acid sequence selected from SEQ ID NO: 1-168. In a preferred embodiment the amino acid sequence is selected from SEQ ID NO: 1, 2, 12, 26, 28, or 32-36.

In another embodiment, the rAAV capsid comprises a binding moiety, wherein the binding moiety comprises or consists of a nucleic acid sequence selected from SEQ ID NO: 169-199 or 201-300. In a preferred embodiment the nucleic acid sequence is selected from SEQ ID NO: 169, 170, 180, 194, 196 or 201-204.

In one embodiment, the rAAV capsid comprises a binding moiety, wherein the binding moiety does not compete with brinzolamide for interacting with carbonic anhydrase 4 (CA4).

In a further embodiment, the rAAV capsid comprises a binding moiety, wherein the binding moiety competes with brinzolamide for interacting with carbonic anhydrase 4 (CA4).

Where herein reference is made to “brinzolamide” it is intended to refer to the compound with IUPAC name (4R)-4-(ethylamino)-2-(3-methoxypropyl)-1,1-dioxo-3,4-dihydrothieno[3,2-e]thiazine-6-sulfonamid, and the molecular formula C₁₂H₂₁N₃O₅S₃. Brinzolamide is known to be an inhibitor of Carbonic Anhydrase 2, but also binds in the catalytic core of CA4 (Shay et al., Sci. Adv. (2023), 9). The catalytic core of CA4 is known to be conserved between species, whereas the surface of CA4 is less conserved. Without being bound by theory, it is expected that rAAV capsids as described herein that compete with brinzolamide for interacting with CA4 will bind CA4 across different species. rAAV capsids that do not compete with brinzolamide for interacting with CA4 are expected to more specifically interact with human CA4 and less or not with murine and/or macaque CA4.

In one embodiment the capacity of the rAAV capsids as described herein to compete with brinzolamide for interacting with CA4 is determined by incubating CA4-expressing cells with brinzolamide and rAAV capsids as described herein, and determining transduction levels of the cells. Preferably, the capacity to compete with brinzolamide for interacting with CA4 is determined using a control rAAV capsid, preferably under otherwise identical conditions.

In one embodiment, the capacity of rAAV capsids as described herein to compete with brinzolamide for interacting with CA4 is determined after 1, 4, 8, 24 or 48 hours of incubation with brinzolamide and the capsids.

In a further embodiment, the transduction levels of CA4-expressing cells with an rAAV capsid as described herein wherein the binding moiety does not compete with brinzolamide for interacting with CA4 are at least 2, 3, 4, 5, 10, 15 or 20 fold higher upon incubation with brinzolamide as compared to a control rAAV capsid.

In one embodiment, the transduction levels of CA4-expressing cells with an rAAV capsid as described herein wherein the binding moiety competes with brinzolamide for interacting with CA4 are up to 1.5 fold higher or up to 2, 3, 4 or 5 fold lower upon incubation with brinzolamide as compared to a control capsid.

### Expression Cassette

In one embodiment, the rAAV capsid comprises an expression cassette flanked by at least one AAV inverted terminal repeat (ITR), wherein the expression cassette comprises a nucleic acid molecule encoding at least one gene product.

Wild type AAV capsids carry a single stranded DNA genome, which consists of two open reading frames carrying the replicase genes and the capsid genes, Rep and Cap, and is flanked by two inverted terminal repeats (ITRs). In recombinant AAV capsids as described herein, the single stranded DNA genome is replaced with an expression cassette.

The term “expression cassette” as used herein describes the functional unit capable of affecting expression of a nucleic acid molecule encoding a gene product. A “nucleic acid molecule encoding a gene product” as referred to herein can be defined as a nucleic acid molecule which comprises a nucleotide sequence encoding at least one gene product.

The nucleic acid molecule encoding a gene product is operably linked to the appropriate expression control sequences, which at least comprises a suitable transcription regulatory sequence and optionally, 3′ transcription termination signals. Additional factors necessary or helpful in affecting expression may also be present, such as expression enhancer elements.

In some embodiments, the nucleic acid molecule encoding a gene product is operably linked to a polyadenylation or polyA or pA signal. The polyadenylation signal aids in the nuclear export of RNA and RNA translation and promotes RNA transcript longevity. In a preferred embodiment, the polyA is located at the 3′ end of the expression cassette upstream of the 3′ ITR.

As used herein, the term “transcription regulatory sequence” or “promoter” refers to a nucleic acid fragment that functions to control the transcription of one or more coding nucleotide sequences, and is located upstream with respect to the direction of transcription of the transcription initiation site of the coding nucleotide sequence. The “transcription regulatory sequence” or “promoter” is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the transcription regulatory sequence or promoter. A “constitutive” transcription regulatory sequence or promoter is a transcription regulatory sequence or promoter that is active under most physiological and developmental conditions. An “inducible” transcription regulatory sequence or promoter is a transcription regulatory sequence or promoter that is physiologically or developmentally regulated, e.g. by the application of a chemical inducer or biological entity.

In one embodiment, the expression cassette is flanked by at least one inverted terminal repeat (ITR).

The term “flanked” with respect to a sequence that is flanked by another element(s) herein indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding a gene product and a flanking element. A sequence that is “flanked” by two other elements (e.g. ITRs), indicates that one element is located 5′ to the sequence and the other is located 3′ to the sequence; however, there may be intervening sequences there between.

In one embodiment, the expression cassette is flanked by two AAV ITR nucleotide sequences, preferably wherein the expression cassette is located in between the two AAV ITR nucleotide sequences, more preferably AAV2 serotype ITR nucleotide sequences or ITR nucleotide sequences which have at least 70%, at least 75%, at least 80%, at least 85%, at least 90%, at least 95%, or more sequence identity with an AAV2 serotype ITR. In another embodiment, the expression cassette is flanked by one ITR engineered with two D regions, wherein preferably the expression cassette is located on either side of the engineered ITR.

In one embodiment, the nucleic acid molecule comprising an expression cassette and at least one ITR as described herein is 5,000 nucleotides (nt) or less in length. The skilled person is aware that the maximum AAV packaging limit is understood to be 5.5 kilobasepairs (kbp). However, in further embodiments, wherein oversized nucleic acid molecules are used, e.g. nucleic acid molecules of more than 5,000 nt in length, or even more than the maximum AAV packaging limit of 5.5 kbp may also still allow incorporation of the nucleic acid molecule in rAAV capsids as described herein and such embodiments are therefore not excluded.

### Promoter

In one embodiment, the expression cassette as described herein comprises a transcription regulatory sequence and/or promoter. In a further embodiment, the transcription regulatory sequence and/or promoter is mammalian cell compatible, preferably the transcription regulatory sequence and/or promoter is operably linked to the nucleotide sequence encoding for at least one product. Many mammalian cell compatible promoters are known in the art (Sambrook and Russell, Molecular Cloning: A laboratory Manual. 3rd Edition, Cold Spring Harbor Laboratory Press, New York, (2001) 1.31-31.42 and 31.59-31.61.).

In one embodiment, the promoter is a constitutive or ubiquitous promoter that is broadly expressed in many cell-types. Exemplary constitutive or ubiquitous promoters include, but are not limited to the Cytomegalovirus (CMV) promoter, the CMV early enhancer/chicken β-actin (CAG) promoter, the chicken β-actin (CBA) promoter and the phosphoglycerate kinase-1 (PGK) promoter.

In a further embodiment, the promoter is an inducible, tissue-specific, cell-type-specific, or cell cycle-specific promoter.

In a preferred embodiment, the promoter is a neuron-specific promoter. Exemplary neuron-specific promoters include, but are not limited to: a neuron-specific enolase (NSE) promoter, platelet-derived growth factor (PDGF) promoter, platelet-derived growth factor B-chain (PDGF-(3) promoter, synapsin or synapsin-1 (Syn or Syn-1) promoter, methyl-CpG binding protein 2 (MeCP2) promoter, Ca+/calmodulin-dependent protein kinase II (CaMKII) promoter, metabotropic glutamate receptor 2 (mGluR2) promoter, neurofilament light (NFL) or heavy (NFH) promoter, β-globin minigene nβ2 promoter, preproenkephalin (PPE) promoter, enkephalin (Enk) promoter and excitatory amino acid transporter 2 (EAAT2) promoter.

In a further embodiment, the promoter is an astrocyte-specific promoter. Exemplary astrocyte-specific promoters include, but are not limited to: glial fibrillary acidic protein (GFAP) and EAAT2 promoters.

In a further embodiment, the promoter is an oligodendrocyte-specific promoter. An exemplary oligodendrocyte-specific promoter includes, but is not limited to the myelin basic protein (MBP) promoter.

In a particularly preferred embodiment, the promoter for expression of gene product in the central nervous system is the CBh promoter (Gray et al., Hum. Gene Ther. 2011, 22:1143-1153).

### Gene Product

As discussed above, in some embodiments, the rAAV capsid as described herein comprises an expression cassette flanked by at least one ITR, wherein the expression cassette comprises a nucleic acid molecule encoding at least one gene product.

The term “gene product” as used herein refers to a nucleic acid molecule encoding at least one RNA molecule and/or encoding at least one polypeptide, or a combination thereof. In a preferred embodiment, the nucleotide sequence encodes a gene product for expression in the mammalian cell and/or encodes a gene product for silencing expression of a gene of interest in a mammalian cell. A particularly preferred mammalian cell in which the “gene product” is to be expressed, is a human cell.

In one embodiment, an expression cassette as described herein may comprise a nucleic acid molecule encoding at least two different gene products, including, but not limited to at least two of the same gene product, or at least two different gene products. Such gene products include, but are not limited to, RNAi agents and polypeptides, as well as combinations thereof.

The term “RNAi agents” as used herein refers to RNA molecules that are capable of RNA interference such as, e.g. a shRNA (short hairpin RNA) a siRNA (short interfering RNA), or a miRNA (microRNA). The term “siRNA” refers to a small interfering RNA that is a short-length double-stranded RNA that can inhibit gene expression in a post-transcriptional matter and are usually not toxic in mammalian cells (Elbashir et al., 2001, Nature 411:494-98; Caplen et al., 2001, Proc. Natl. Acad. Sci. USA 98:9742-47). Similarly to siRNAs, miRNAs also inhibit gene expression in a post-transcriptional manner. Although the gene silencing effects of siRNAs and miRNAs are distinct, the distinction has been obscured because they are associated with common enzymes (e.g., Dicer and RISC) and their functions overlap with each other to a certain extent. The major difference between siRNAs and miRNAs is that the former inhibit the expression of one specific target mRNA while the latter regulate the expression of multiple mRNAs (Lam et al., 2015, Mol Ther Nucleic Acids. 2015 Sep. 15; 4(9): e252).

In one embodiment, the gene product as described herein is a therapeutic gene product. The term “therapeutic gene product” as used herein can refer to a polypeptide, or an (non-coding) RNA molecule (si/sh/miRNA), or other gene product that, when expressed in a target cell, provides a desired therapeutic effect. A desired therapeutic effect may be the ablation of an undesired activity, the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof.

In one embodiment, there is provided an rAAV capsid as described herein, comprising an expression cassette flanked by at least one ITR, wherein the expression cassette comprises a nucleic acid molecule encoding at least one gene product having an effect on an CNS disease.

In one embodiment, there is provided an rAAV capsid as described herein, comprising an expression cassette flanked by at least one ITR, wherein the expression cassette comprises a nucleic acid molecule encoding an RNA molecule and/or polypeptide having an effect on an CNS disease. In a preferred embodiment, an RNA molecule and/or polypeptide as described herein slows down the progression of a CNS disease. In a further embodiment, an RNA molecule and/or polypeptide as described herein, slows down the deterioration of the condition of a subject suffering from a CNS disease. In one embodiment, an RNA molecule and/or polypeptide as described herein eliminates one or more symptoms of a CNS disease.

In one embodiment, the expression cassette may comprise a nucleotide sequence encoding a polypeptide that serves as a selection marker protein to assess cell transformation and expression. Suitable marker proteins for this purpose include, but are not limited to: the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene.

### Polynucleotides

In a second aspect, there is provided a polynucleotide encoding an rAAV capsid as described herein.

The term “polynucleotide” as used herein refers to a nucleic acid molecule comprising a multitude of nucleotides. The terms “polynucleotide” may be used interchangeably with the term “nucleic acid molecule”.

### Host Cell

In a third aspect, there is provided a host cell comprising a polynucleotide for expression of the rAAV capsid as described herein.

In a preferred embodiment, the polynucleotide is an expression vector for expression of an rAAV capsid as described herein.

In one embodiment, the expression vector for expression of the rAAV capsid as described herein is an insect cell-compatible vector or a mammalian cell-compatible vector. A “mammalian cell-compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses. Any vector can be employed as long as it is insect cell- or mammalian cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, infection or transduction. The expression vector will be introduced into a suitable host cell and be able to effect expression of the coding sequence in an in vitro cell culture of the host cell. A preferred expression vector will be suitable for expression of viral proteins and/or nucleic acids, particularly recombinant parvoviral proteins and/or nucleic acids, such as baculoviral vectors for expression of parvoviral proteins and/or nucleic acids in insect cells.

A host cell as described herein may be any cell that is suitable for the production of heterologous proteins. In one embodiment, the host cell as described herein is an insect cell, more preferably, an insect cell that allows for replication of baculoviral vectors and can be maintained in culture. In a preferred embodiment, the insect cell allows for expression of at least one baculoviral expression vector such that recombinant parvoviral vectors, including rAAV vectors are produced. Exemplary host cells include, but are not limited to cells derived from: Spodoptera frugiperda, Drosophila cell lines, or mosquito cell lines, including Aedes albopictus-derived cell lines. In a preferred embodiment, the host cells are derived from insect species which are susceptible to baculovirus infection, including, but not limited to: S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA). In a preferred embodiment, a host cell as described herein is an insect cell for production of rAAV capsids, preferably an expresSF+® cell.

In one embodiment, the host cell as described herein is a mammalian cell, more preferably, a mammalian cell that allows for expression and/or replication of mammalian cell-compatible expression vectors and can be maintained in culture. Exemplary mammalian cell lines for producing AAV vectors include, but are not limited to: A549, WEHI, 3T3, 10T1/2, BHK, MDCK, COS 1, COS 7, BSC 1, BSC 40, BMT 10, VERO, WI38, HeLa, a HEK 293 cell, Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this disclosure; nor is the type of mammalian cell, i.e., fibroblast, hepatocyte, tumour cell. Mammalian cell lines for producing rAAV capsids in particular include a broad range of HEK293 cell lines. In a preferred embodiment, a mammalian cell as described herein is a mammalian cell for production of rAAV capsids. In one embodiment, there is provided the polynucleotide as described herein in a host cell, preferably in an mammalian cell. In a preferred embodiment, the host cell is a HEK293T cell.

In one embodiment, there is provided at least one expression vector comprising separate expression cassettes for each of the VP1, VP2 and VP3 capsid proteins, which capsid proteins, independently may be a variant, comprising a binding moiety, as described herein. In one embodiment, there is provided at least one expression vector comprising a separate expression cassette for a VP1 capsid protein and a separate expression cassette for the VP2 and VP3 proteins, wherein the VP1 capsid protein and/or both VP2 and VP3 capsid proteins, may be variants as described herein. The various expression cassettes for the different capsid protein variants as described herein can be present together on a single expression vector, or one or more of the expression cassettes for the different capsid proteins can be present on two or three separate expression vectors, each comprising one of the expression cassettes for the different capsid proteins. As will be understood, the use of separate expression cassettes for one or more of the different capsid proteins allows to produce rAAV capsids with an above-described modification/mutation in only some of capsid proteins, e.g. only in VP1 and not in VP2 and VP3 or vice versa, or it allows to produce rAAV capsids with different above-described modifications/mutations in the different capsid proteins. Expression vectors for separate expression of the various capsid proteins in mammalian cells are e.g. disclosed in Judd et al. (Mol Ther Nucleic Acids. 2012; 1: e54). Expression vectors for separate expression of the various capsid proteins in insect cells are disclosed in WO2022/253955.

The term “variant” as used herein refers to a nucleic acid sequence or amino acid sequence comprising one or more alterations as compared to a known or described reference sequence. In some embodiments, a variant sequence encodes a protein with altered characteristics or functionality as compared to the reference sequence. In other embodiments, a variant sequence encodes a protein with similar or the same functionality as compared to the reference sequence. In preferred embodiments, a variant has at least 70%, 75%, 80%, 85%, 90%, 95%, 96%, 97%, or 98%, preferably 99% sequence identity to a reference sequence.

In a preferred embodiment, there is provided an expression vector comprising a single expression cassette for expression of all three of the VP1, VP2 and VP3 capsid protein variants, comprising a binding moiety, as described herein, preferably from a single expression cassette. Expression vectors for expression of all three of the VP1, VP2 and VP3 capsid proteins from a single expression cassette in mammalian cells are e.g. disclosed in Clark et al. (Hum. Gene Ther. 1995, 6, 1329-134), Gao et al. (Hum. Gene Ther. 1998, 9, 2353-2362), Inoue and Russell (J. Virol. 1998, 72, 7024-7031), Grimm et al. (Hum. Gene Ther. 1998, 9, 2745-2760) and Xiao et al. (J. Virol. 1998, 72, 2224-2232). Expression vectors for expression of all three of the VP1, VP2 and VP3 capsid proteins from a single expression cassette in insect cells are e.g. disclosed in Urabe et al. (Hum. Gene Ther. 2002, 13:1935-1943), WO2007/046703, WO2015/137802 and WO2019/016349. As will be understood, expression of all three of the VP1, VP2 and VP3 capsid protein variants as described herein, from a single expression cassette allows to produce rAAV capsids comprising mutations/modifications as described herein in all three of its VP1, VP2 and VP3 capsid proteins.

In one embodiment, an expression vector for expression of an rAAV capsid as described herein comprises a nucleic acid comprising a suboptimal translation initiation codon for the VP1 capsid protein, wherein the suboptimal translation initiation codon is selected from CTG, ACG, TTG or GTG. In some embodiments, the suboptimal translation initiation codon is followed by an additional second codon encoding an amino acid selected from alanine, glycine, valine, aspartic acid and glutamic acid, preferably valine or alanine.

In one embodiment, the host cell further comprises an expression vector encoding an expression cassette flanked by at least one ITR, wherein the expression cassette comprises a nucleic acid molecule encoding at least one gene product.

### Composition

In a fourth aspect, there is provided a composition comprising an rAAV capsid as described herein, preferably wherein the composition is a pharmaceutical composition further comprising at least one pharmaceutically acceptable carrier.

In one embodiment, there is provided a composition comprising an rAAV capsid as described herein and a suitable excipient. Suitable excipients include, but are not limited to: a buffer, stabilizer, an antioxidant. In preferred embodiments the composition is used for treatment of (human) subjects. In some embodiments, the pharmaceutical composition comprises physiological buffers for buffering the composition to a pH of about 7.3-7.5. Exemplary physiological buffers include, but are not limited to: phosphate buffered saline (PBS), or tris(hydroxymethyl)aminomethane (Tris). Furthermore, a pharmaceutical composition may comprise further stabilizing agents including, but not limited to: sucrose, mannitol, trehalose, cyclodextrin, sodium chloride, potassium chloride, hydroxypropyl-beta-cyclodextrin (HP-BCD), or polysorbate. In a preferred embodiment, the pharmaceutical composition is suitable for systemic administration. More preferably the pharmaceutical composition is suitable for systemic administration using injection and/or infusion. In one embodiment, the pharmaceutical composition is suitable for intravenous and/or intraarterial injection and/or infusion.

rAAV Capsid or Composition for Use

In a fifth aspect there is provided an rAAV capsid as described herein, or a composition as described herein, for use as a medicament, preferably for use in the treatment of a condition of the central nervous system.

In one embodiment, there is provided a method of treating a condition of the CNS by administering a rAAV capsid as described herein, or composition as described herein.

In one embodiment, there is provided the use of a rAAV capsid as described herein in the manufacture of a medicament for the treatment of a condition of the CNS.

### Conditions of the Central Nervous System (CNS)

The terms “Central Nervous System” and “CNS”, as used herein refer to both the brain and the spinal cord.

The term a “condition of the central nervous system (CNS)” as used herein refers to a condition wherein the brain and/or spinal cord do not exert their function as compared to the brain and/or spinal cord of a healthy individual. The terms “a condition of the CNS”, “disease of the CNS” or “CNS disease” or “CNS disorder” may be used interchangeably. Diseases that also affect the brain and/or spinal cord next to having different symptoms can also be a “disease of the CNS”, or “CNS disease”. Such diseases include, for instance, neurological diseases which affect both the central nervous system and the peripheral nervous system.

In one embodiment, the brain and/or spinal cord function is impaired as compared to a healthy individual in a condition of the CNS as defined herein.

In a preferred embodiment, the condition of the CNS is caused by a genetic defect.

In a further embodiment, the genetic defect is a mutation in a gene and/or accumulation of dysfunctional or non-functional proteins when compared to a healthy individual.

Exemplary conditions of the CNS include, but are not limited to: Parkinson's Disease (PD); Parkinson disease 1, autosomal resistant (PARK1); Parkinson disease 4, autosomal dominant (PARK4); Multiple System Atrophy (MSA); Lewy Body Dementia (LBD); Huntington's Disease (HD); Spinocerebellar Ataxia 1 (SCA1); Spinocerebellar Ataxia 2 (SCA2); Spinocerebellar Ataxia 3 (SCA3); Primary Tauopathies; Pick's Disease (PiD); Progressive Supranuclear Palsy (PSP); Corticobasal Degeneration (CBD); Argyrophilic Grain Disease (AGD); Secondary Tauopathies; Chronic Traumatic Encephalopathy (CTE); Alzheimer's Disease (AD); Frontotemporal Dementia (FTD); Amyotrophic Lateral Sclerosis (ALS); Temporal Lobe Epilepsy (TLE); refractory Temporal Lobe Epilepsy (rTLE); Focal Cortical Dysplasia (FCD); Focal Cortical Dysplasia 2 (FCORD2); Smith-Kingsmore Syndrome (SKS); Spinal Muscular Atrophy 1 (SMA1; Werdnig-Hoffmann disease); Spinal Muscular Atrophy 2 (SMA2, Dubowitz disease); Spinal Muscular Atrophy 3 (SMA3, Kugelberg-Welander disease); Spinal Muscular Atrophy 4 (SMA4); Gaucher Disease (GD); Mucopolysaccharidosis 3A (MPS3A); Mucopolysaccharidosis 2 (MPS2); Mucopolysaccharidosis 1 (MPS1); Ceroid Lipofuscinosis, Neuronal, 1 (CLN1); Immunodeficiency 78 with autoimmunity and developmental delay (IMD78); Ceroid Lipofuscinosis, Neuronal, 3 (CLN3); Batten Disease; Ceroid Lipofuscinosis, Neuronal, 5 (CLN5); Ceroid Lipofuscinosis, Neuronal, 6 (CLN6); Ceroid Lipofuscinosis, Neuronal, 7 (CLN7); Ceroid Lipofuscinosis, Neuronal, 8 (CLN8); Canavan Disease (CAND); Ubiquitin-positive Frontotemporal Dementia (UP-FTD); Ceroid Lipofuscinosis, Neuronal, 11 (CLN11); Angelman Syndrome (AS); Intellectual Developmental Disorder, X-linked, Syndromic 13 (MRXS13); Rett Syndrome (RTT); Autism, X-linked 3 (AUTSX3); Encephalopathy, Neonatal Severe, due to MECP2 Mutations (ENS-MECP2); Intellectual Developmental Disorder, X-linked, Syndromic, Lubs type (MRXSL); GM1-Gangliosidosis 1 (GM1G1); GM1-Gangliosidosis 2 (GM1G2); GM1-Gangliosidosis 3 (GM1G3); Giant Axonal Neuropathy 1; Autosomal Recessive (GAN1); Citrullinemia (CTLN1); GM2-Gangliosidosis 1 (GM2G1); Tay-Sachs Disease; Fragile X Syndrome (FXS); Fragile X tremor/ataxia Syndrome (FXTAS); Cerebral Cavernous Malformations 4 (CCM4); Epilepsy, familial focal, with variable foci 1 (FFEVF1); Niemann-Pick Disease A (NPDA); Niemann-Pick Disease C (NPDC); Krabbe Disease (KRB); Mucopolysaccharidosis 1H (MPS1H); Epilepsy Metachromatic Leukodystrophy (MLD); Multiple Sulfatase Deficiency (MSD); Ceroid Lipofuscinosis, Neuronal, 2 (CLN2); Friedreich Ataxia (FRDA); Tuberous Sclerosis; Aromatic L-Amino Acid Decarboxylase Deficiency (AADCD); Pompe Disease; Mucopolysaccharidosis 3B (MPS3B); Alexander disease (ALXDRD); Dup15q syndrome; Developmental and epileptic encephalopathy 79 (DEE79); Epilepsy, childhood absence 5 (ECA5); Developmental and epileptic encephalopathy (DEE43); Prader-Willi syndrome (PWS); Phelan-McDermid Syndrome (PHMDS; PMS), Dravet Syndrome (DRVT); Lafora Disease (LD); Epilepsy, progressive myoclonic 2 (EPM2); Familial Hemiplegic Migraine 3 (FHM3); Generalized epilepsy with febrile seizures plus 2 (GEFSP2); Intractable childhood epilepsy with generalized tonic-clonic seizures (ICEGTC); Febrile seizures, familial, 3A (FEB3A); Developmental and epileptic encephalopathy 6B (DEE6B); Familial Hemiplegic Migraine 1 (FHM1); Episodic ataxia 2 (EA2); Developmental and epileptic encephalopathy 42 (DEE42); Familial Hemiplegic Migraine 2 (FHM2); Alternating hemiplegia of childhood 1 (AHC1); Fetal akinesia, respiratory insufficiency, microcephaly, polymicrogyria and dysmorphic facies (FARIMPD); Developmental and epileptic encephalopathy 98 (DEE98).

In a preferred embodiment, the condition of the CNS is selected from: Huntington's Disease (HD), Amyotrophic Lateral Sclerosis (ALS); Temporal Lobe Epilepsy (TLE); refractory Temporal Lobe Epilepsy (rTLE); Focal Cortical Dysplasia (FCD); Parkinson's Disease (PD); Parkinson disease 1, autosomal resistant (PARK1); Parkinson disease 4, autosomal dominant (PARK4); Multiple System Atrophy (MSA), Alzheimer's Disease (AD); Fragile X Syndrome (FXS); Fragile X tremor/ataxia Syndrome (FXTAS); and Dup15q syndrome.

In one embodiment, the rAAV capsid is administered systemically, preferably intravenously or intraarterially. In a preferred embodiment, the rAAV capsid is administered intravenously or intraarterially at a site close to the BBB.

### Gene Therapy

In a sixth aspect, there is provided a rAAV capsid as described herein, or a composition as described herein, for use in gene therapy.

The term “gene therapy” as used herein refers to a therapy that uses one or more gene products as described herein to treat, prevent or cure a disease or medical disorder. In one embodiment, the treatment, prevention or cure is long-lasting and sustained and follows upon a single administration of the gene therapy.

In one embodiment, there is provided a method of gene therapy comprising the step of administering an effective amount of an rAAV capsid as described herein, or a composition comprising the rAAV capsid as described herein, to a subject in need of gene therapy.

In one embodiment, the gene therapy is for the treatment of a condition of the CNS as defined herein above.

### Method of Manufacturing

In a seventh aspect, there is provided a method of manufacturing an rAAV capsid as described herein, the method comprising the steps of: I) culturing a host cell comprising: i) an expression vector for expression of a rAAV capsid as described herein, and; ii) an expression vector encoding an expression cassette flanked by at least one AAV inverted terminal repeat (ITR), comprising a nucleic acid molecule encoding at least one gene product; said host cell preferably further comprising a nucleotide sequence encoding a parvoviral replication (Rep) protein under conditions such that the rAAV capsid is produced; and, II) recovery of the rAAV capsid, preferably wherein recovery of the rAAV capsid comprises at least one of: affinity-purification of the rAAV capsid using an immobilized anti-AAV antibody, preferably a single chain camelid antibody or a fragment thereof, or filtration using a filter having a nominal pore size of 30-70 nm.

In general, suitable methods for producing an rAAV capsid as described herein in mammalian or insect host cells, and means therefore (such as expression constructs for expression of AAV rep proteins), are described, for mammalian cells in: Clark et al. (Hum. Gene Ther. 1995, 6, 1329-134), Gao et al. (Hum. Gene Ther. 1998, 9, 2353-2362), Inoue and Russell (J. Virol. 1998, 72, 7024-7031), Grimm et al. (Hum. Gene Ther. 1998, 9, 2745-2760), Xiao et al. (J. Virol. 1998, 72, 2224-2232) and Judd et al. (Mol Ther Nucleic Acids. 2012; 1: e54), and for insect cells in: Urabe et al. (Hum. Gene Ther. 2002, 13:1935-1943), WO2007/046703, WO2007/148971, WO2009/014445, WO2009/104964, WO2011/122950, WO2013/036118, WO2015/137802, WO2019/016349, WO2021/198508, WO2021/198510 and WO2022/253955, all of which are incorporated herein in their entirety.

AAV sequences that may be used as described herein for the production of an rAAV capsid as described herein in insect cells can be derived from the genome of any AAV serotype. Generally, the AAV serotypes have genomic sequences of significant homology at the amino acid- and nucleic acid levels, provide an identical set of genetic functions, and produce capsids which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (J. Vir. 1997, 71:6823-33); Srivastava et al. (J. Vir. 1983, 45:555-64); Chlorini et al. (J. Vir. 1999, 73:1309-1319); Rutledge et al. (J. Vir. 1998, 72:309-319); and Wu et al. (J. Vir. 2000, 74:8635-47). Preferably the AAV ITR sequences for use in the context of the present invention are derived from AAV1, AAV2, AAV4 and/or AAV7, more preferably AAV2. Likewise, the Rep (Rep78/68 and Rep52/40) coding sequences are preferably derived from AAV1, AAV2, AAV4 and/or AAV7.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., J. Virol., 1999, 73 (2): 939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells. Modified “AAV” sequences also can be used in this context, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity to an AAV1, AAV2, AAV3, AAV4, AAV5, AAV6, AAV7, AAV8, AAV9, AAV10, AAV11, AAV12 or AAV13 ITR or Rep can be instead of place of wild-type AAV ITR or Rep sequences.

In one embodiment, a mammalian cell for producing an rAAV capsid as described herein is selected from cell lines among any mammalian species, including, but not limited to: A549, WEHI, 3T3, 10T1/2, BHK, MDCK, COS 1, COS 7, BSC 1, BSC 40, BMT 10, VERO, WI38, HeLa, a HEK 293 cell, Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this disclosure; nor is the type of mammalian cell, i.e., fibroblast, hepatocyte, tumour cell. Mammalian cell lines for producing rAAV capsids in particular include a broad range of HEK293 cell lines, of which the HEK293T cell line is preferred.

Insect cell lines for producing an rAAV capsid as described herein can be any cell line that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, including Aedes albopictus-derived cell lines. In a preferred embodiment, the insect cell or cell line is derived from insect species which are susceptible to baculovirus infection, including but not limited to: S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

The rAAV capsids that are released into the supernatant of the mammalian or insect cell culture can be recovered and/or purified using suitable techniques which are known to those of skill in the art. In one embodiment, monolith columns (e.g., in ion exchange, affinity or IMAC mode), chromatography (e.g., capture chromatography, fixed method chromatography, and expanded bed chromatography), centrifugation, filtration and/or precipitation, are used for purification and concentration. These methods may be used alone or in combination. In one embodiment, capture chromatography methods, including column-based or membrane-based systems, are utilized in combination with filtration and precipitation. Suitable precipitation methods, including, but not limited to, chromatography methods utilizing polyethylene glycol (PEG) 8000 and NH3SO4, can be readily selected by one of skill in the art. Thereafter, the precipitate can be treated with enzymes, e.g. nucleases such as benzonase and purified using suitable techniques. In addition, recovery preferably comprises the step of affinity-purification of the rAAV capsid using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, Biotechnol. 2001, 74:277-302). The antibody for affinity-purification of rAAV capsids preferably is an antibody that specifically binds an epitope on an rAAV capsid protein, whereby preferably the epitope is an epitope that is present on capsid protein of more than one AAV serotype. E.g. the antibody may be raised or selected on the basis of specific binding to AAV6 capsid but at the same time also it may also specifically bind to rAAV9 capsids.

### DESCRIPTION OF THE FIGURES

FIG. 1. Schematic diagram depicting the generation of rAAV capsids with different binding moieties.

A) pIMRAC (Insert-cell & MRna-based Aav Capsid library) library backbone comprising a randomly degenerated sequence (rainbow box). B) Rolling Circle amplification and TelN digestion: amplification of the library and generation of suitable templates for rAAV library production. C) Transfection and baculovirus infection of cells, generating rAAV capsids.

FIG. 2. Screening for hCA4-interacting capsid variants.

A) In vitro screening of capsid library. B) Analysis of the capsid cDNA output by next-generation sequencing (NGS) for random codon/peptide enrichment within the VR-VIII loop. Distribution of the random peptide library is mapped in comparison to the β capsid library input and the capsid library output from in vitro evolution in naïve HEK293T cells. C) Capsid variants found to be enriched in hCA4 overexpressing cells only.

FIG. 3. Second Screening for hCA4-Interacting capsid variants.

A) In vitro screening of capsid library with transiently CA4 overexpressing cells. B) NGS evaluation of sorted cells for capsid library enrichment. C) Capsid variants found to be enriched in hCA4 overexpressing cells only.

FIG. 4. Motif Sequences of Peptide Ligands Displayed on hCA4-Interacting Capsid Variants.

A) Sequence alignment of peptide hits from both the first and second screen. B) Phylogenetic analysis of the total hCA4-engaging capsid variants.

FIG. 5. Schematic Diagram Illustrating the Single-Plex Method for Hit Discovery and Identification of AAVCapX Hits Interacting with the hCA4 Receptor.
A) Capsid variant expression in insect cells. B) Assessment of Nluc expression in cells harbouring CA4 or a mock receptor. C) Fold enrichment of the different capsid variants for different CA4 receptor variants (murine, macaque, human). D) Capsid variants with highest enrichment scores for human CA4.

FIG. 6. Dose-Range Confirmation of hCA4-Interacting AAVCapX Hits.

A) Ratio of murine CA4 receptor reporter (Nluc) over mock receptor reporter (Fluc) for five CapX variants, compared to two control capsids, at increasing MOI (5E+2, 5E+3, or 5E+4 GC/cell). B) Ratio of human CA4 receptor reporter (Nluc) over mock receptor reporter (Fluc) for five CapX variants, compared to two control capsids, at increasing MOI (5E+2, 5E+3, or 5E+4 GC/cell).

FIG. 7. Dose-Range Confirmation of hCA4-Interacting AAVCapX Hits.

A) Ratio of mock receptor over mock receptor and brinzolamide (100 nM) (Fluc) for five CapX variants, compared to two control capsids, at increasing MOI (5E+2, 5E+3, or 5E+4 GC/cell). B) Ratio of murine CA4 receptor over murine CA4 receptor and brinzolamide (100 nM) (Fluc) for five CapX variants, compared to two control capsids, at increasing MOI (5E+2, 5E+3, or 5E+4 GC/cell). C) Ratio of human CA4 receptor over human CA4 receptor and brinzolamide (100 nM) (Fluc) for five CapX variants, compared to two control capsids, at increasing MOI (5E+2, 5E+3, or 5E+4 GC/cell).

### EXAMPLES

### Introduction

In this study, a library and screening platform was used to generate and screen for rAAV capsids capable of utilizing human Carbonic anhydrase IV (hCA4) to cross the BBB and achieve widespread brain transduction via systemic administration. Conventional capsid library evolution strategies are less suited to this end as they would yield capsid candidates with random properties. Therefore and in vitro screening strategy was used to simplify and rationally bias the capsid library evolution towards hCA4 usage.

### 1. Example 1: Generation of the Capsid Library and its Application in In Vitro Screening for hCA4-Dependent BBB-Penetrating Capsids

### 1.1 Methods

### 1.1.1 BRAIN-X AAV Capsid Library Production

DNA libraries were constructed by cloning double-stranded DNA fragments into a digested recipient pIMRAC plasmid using the Gibson assembly method (NEBuilder HiFi DNA Assembly Master Mix). Plasmid propagation was performed using rolling circle amplification (RCA) with EquiPhi29™ DNA Polymerase (ThermoFisher). The resulting DNA product was digested with TelN protelomerase, which targets TelN recognition sites in the plasmid backbone.

The resulting DNA was transfected into Sf+ cells at a ratio of 0.07 pg per cell. Three days post-transfection, the cells were infected with a baculovirus encoding AAV replicase. Seventy-two hours post-infection, the cells were lysed, and the rAAV capsid library was harvested and purified using POROS CaptureSelect AAVX Affinity Resin.

The rAAV capsid library was then titrated using qPCR with primers/probes binding to the hr4b enhancer. The presence and relative abundance of viral proteins were analyzed by SDS-PAGE. Additionally, viral DNA was isolated from the purified product using the PureLink Viral RNA/DNA Mini Kit and used as a template to amplify the library insertion site for Sanger sequencing, confirming the transfer of the library from the cloned plasmid to the final capsid library.

### 1.2 Results

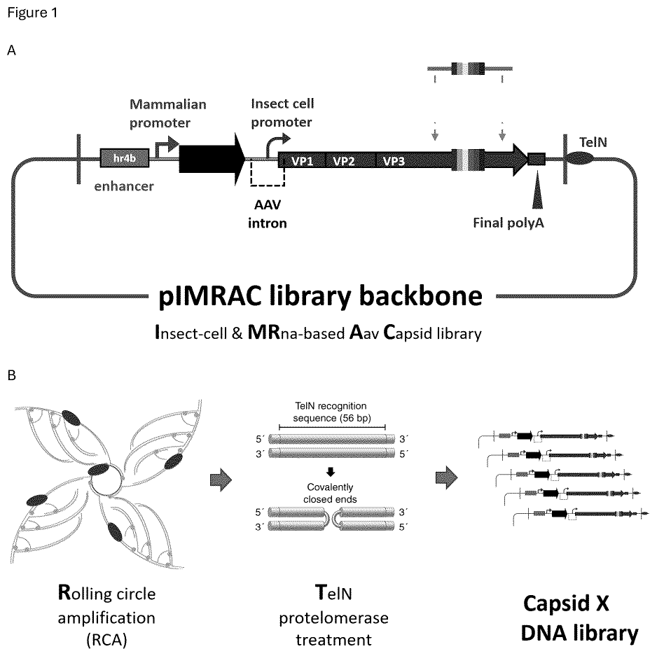

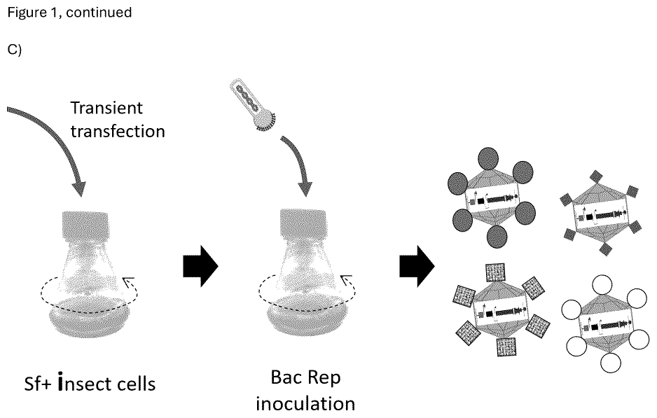

A DNA fragment with a randomly degenerated sequence (indicated as a rainbow box in FIG. 1A) was synthesized and cloned into the VR-VIII loop of a rAAV9 capsid sequence [(capctrl)-SEQ ID NO: 200] in plMRAC (Insect-cell & mRNA-based AAV Capsid library) (FIG. 1A). Two versions of the library were created: Library #1 contains a 7x degenerated NNK codon sequence between Q588 and A596, while Library #2 includes codons encoding D587, G588, and P589, followed by 5× degenerated NNK codons and R595. Immediately after cloning, the DNA mixture was subjected to rolling circle DNA amplification (RCA) and TelN digestion to generate the capsid-X DNA genome library (FIG. 1B). This library was then used in transfection and infection experiments (FIG. 1C). Following baculovirus Rep (Bac Rep) transactivation, the transfected insect cells produced rAAV capsids. These capsids have random peptide insertions in the VR-VIII loop derived from the DNA genome previously transfected into the cells, and the same DNA genome is also encapsidated by the newly produced capsids.

### 2. Example 2: Confirmation of Library Hit Interactions with hCA4 Via a Single-Plex Discovery Assay

### 2.1 Methods

2.1.1 rAAV Capsid Library In Vitro Screening

The rAAV capsid libraries were screened in vitro using an in-house developed human carbonic anhydrase IV (hCA4) expressing cell line with a HEK293T background. Cells were transduced with the rAAV capsid library at an MOI of 1e5 and co-infected with wild-type adenovirus 5 (Ad5). After 72 hours, cells were lysed, and total RNA was isolated using the AllPrep DNA/RNA Mini Kit. Messenger RNA (mRNA) was enriched using the QIAGEN RNeasy Pure mRNA Bead Kit and used for cDNA synthesis followed by PCR amplification of the library insertion site. Sanger sequencing was employed to evaluate the presence of the library in the resulting PCR product, which was subsequently re-cloned into the vector plasmid to produce the β capsid library for a second in vitro iteration.

2.1.2 Second rAAV Capsid Library In Vitro Screening

The rAAV capsid libraries were screened in vitro using transiently overexpressing hCA4 cell line with a HEK293T background. Cells were transduced with the rAAV capsid library at an MOI of 1e5 and co-infected with wild-type adenovirus 5 (Ad5). After 72 hours, cells were Fluorescence-activated Cell Sorting (FACS) sorted, lysed, and total RNA was isolated using the AllPrep DNA/RNA Mini Kit. Messenger RNA (mRNA) was enriched using the QIAGEN RNeasy Pure mRNA Bead Kit and used for cDNA synthesis followed by PCR amplification of the library insertion site. Sanger sequencing was employed to evaluate the presence of the library in the resulting PCR product, which was subsequently re-cloned into the vector plasmid to produce the β capsid library for a second in vitro iteration.

### 2.1.3 NGS Sample Preparation, Sequencing, and Analysis

After in vitro screening, the fragment containing the library was PCR amplified from: 1) the β capsid library used for transduction in the second iteration (input), 2) the mRNA from hCA4-expressing cells transduced with the β capsid library, and 3) the mRNA from HEK293T (non-hCA4-expressing) cells transduced with the β capsid library. The PCR amplicons were used for Illumina library preparation (TruSeq DNA Nano Kit), and the resulting libraries were sequenced on the NovaSeq6000 sequencer with paired-end reads of 151 nt, aiming for at least 5 million paired-end reads per sample.

Paired reads were merged with Pear (v0.9.6) and translated into all possible reading frames using seqtk. An in-house developed Python script was used to identify the amino acid and nucleotide sequences of the capsid variants and their abundance in each sample. Raw counts of each variant were normalized to counts per million (CPM) to account for differences in sequencing depth across samples. The enrichment score of each capsid variant in the hCA4-expressing cells was calculated by dividing the CPM in hCA4-expressing cells by the CPM in the input β capsid library. Similarly, to account for hCA4-binding specificity, the CPM of each capsid variant in the hCA4-expressing cells was divided by the CPM in HEK293T cells (naïve cells). For hit selection, capsid variants that were enriched ≥10-fold in hCA4 relative to the β capsid library input and >5-fold relative to HEK293T naïve cells were cloned into a plasmid backbone suitable for small-scale production in Sf+ cells and single-plex validation.

### 2.1.4 Single-Plex Hit Discovery Assay

HEK293T cells were seeded and reverse transfected with either a plasmid (pOX) encoding hCA4, mScarlet, and NLuc reporters, or with a plasmid expressing both mScarlet and firefly luciferase (FLuc) reporters but with a mock receptor. Two days after transfection, cells were transduced with each capsid variant. Forty-eight hours post-transduction, cells were lysed, and FLuc and NLuc were measured using the Nano-Glo Dual-Luciferase Reporter Assay System. The enrichment score of each capsid variant in the hCA4-transfected cells was calculated by dividing the NLuc readout in the hCA4-transfected cells relative to the NLuc readout in cells transfected with an unrelated receptor. Initially, cells were transduced with a single (volumetric) MOI; subsequently, cells were transduced with three increasing MOIs (5E+2, 5E+3, or 5E+4 GC/cell). All experiments were performed in triplicate with n=2.

### 2.2 Results

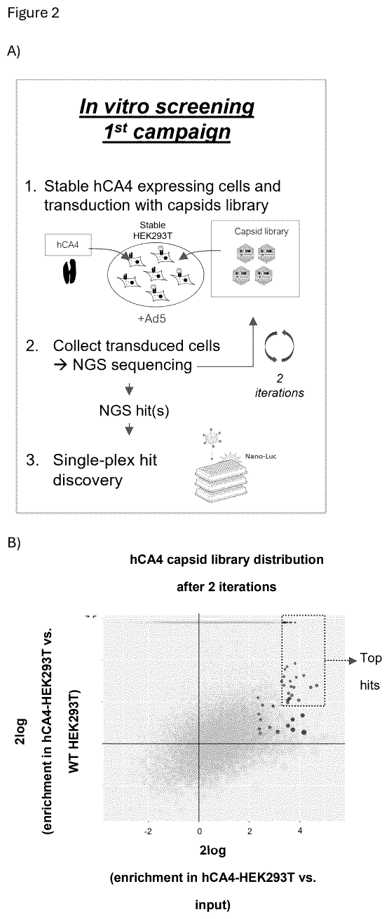

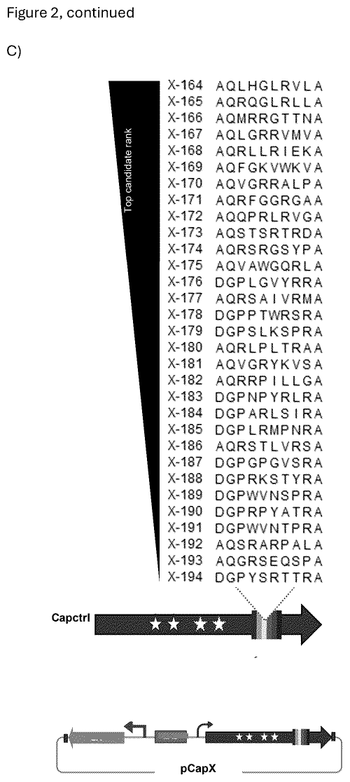

HEK293T cells stably overexpressing hCA4 were transduced with the 1st generation (a) capsid library and wild-type adenovirus (Ad5) (FIG. 2A). The use of Ad5 amplified the cDNA expression from the capsid genome library in the target cells, which was then used to generate the 2nd generation (B) capsid library for the next iteration. After 2 iterations, the capsid cDNA output was analyzed by next-generation sequencing (NGS) for random codon/peptide enrichment within the VR-VIII loop. During enrichment analysis, the distribution of the random peptide library was mapped in comparison to the β capsid library input and the capsid library output from in vitro evolution in naïve HEK293T cells. As a result, several variants of AAV9 harboring random peptide insertions were observed to be enriched in hCA4 overexpressing cells only (FIGS. 2B and C).

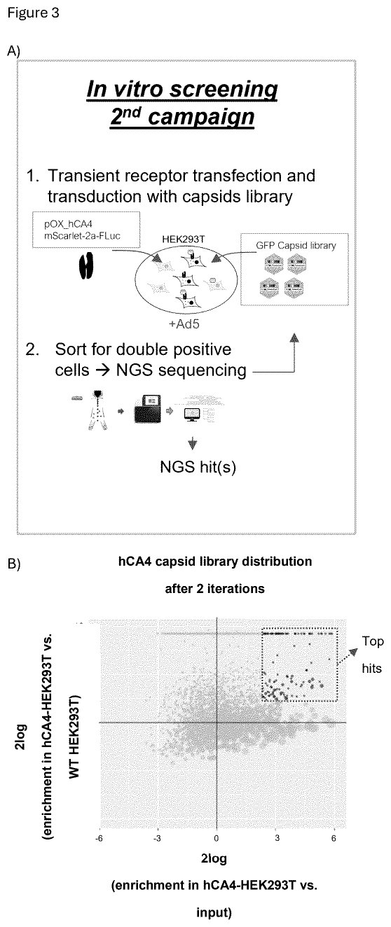

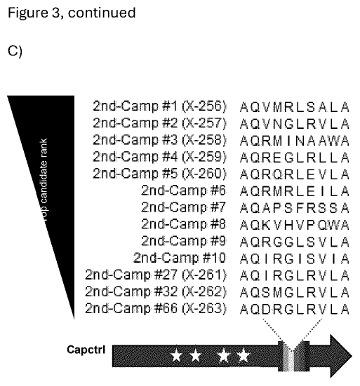

Next the consistency of the library approach in targeting the same hCA4 receptor was evaluated by conducting a 2nd campaign. To simplify the workflow, stable cells overexpressing the target transmembrane receptor were replaced with transiently overexpressing cells via pOX transfection, followed by FACS sorting strategy (FIG. 3A). By incorporating a GFP reporter into the pIMRAC library backbone (FIG. 1A) and utilizing mScarlet expression within the pOX (FIG. 3B), we were able to sort double GFP and mScarlet positive cells. These sorted cells were then evaluated for capsid library enrichment using a similar NGS-based analysis as in the 1st campaign.

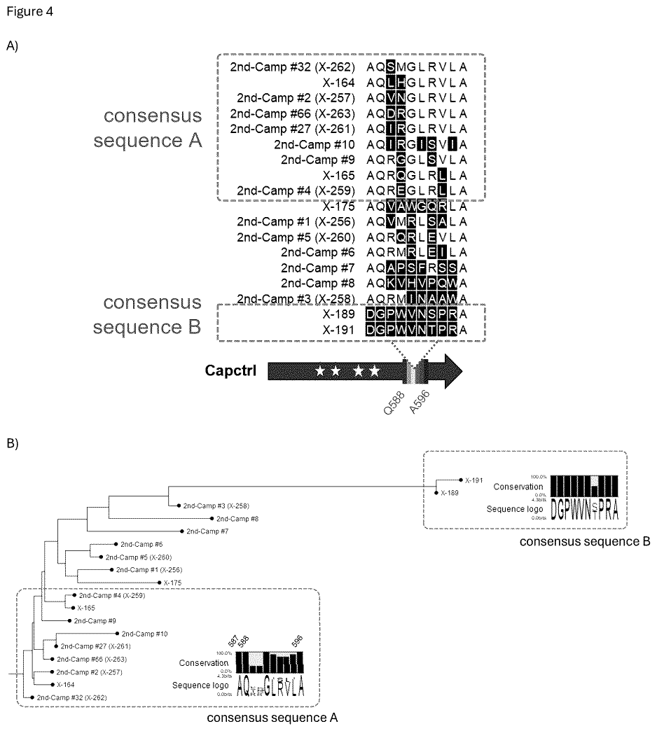

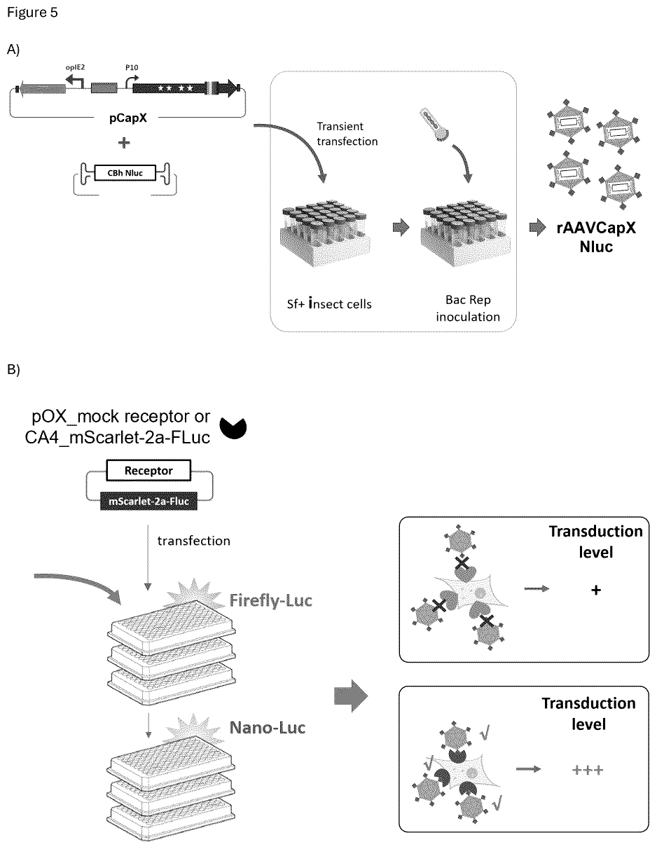

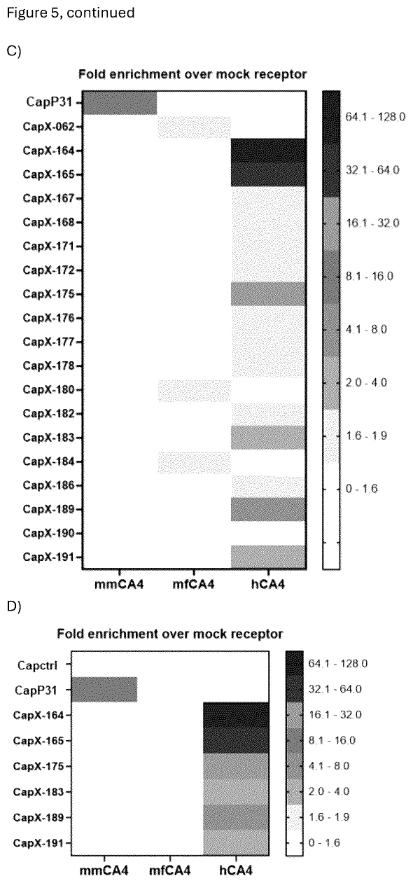

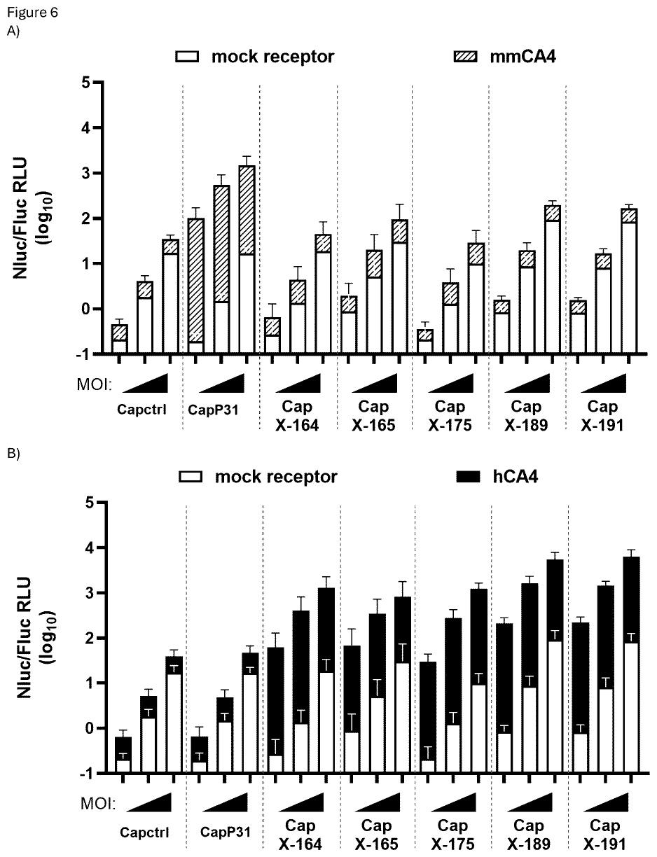

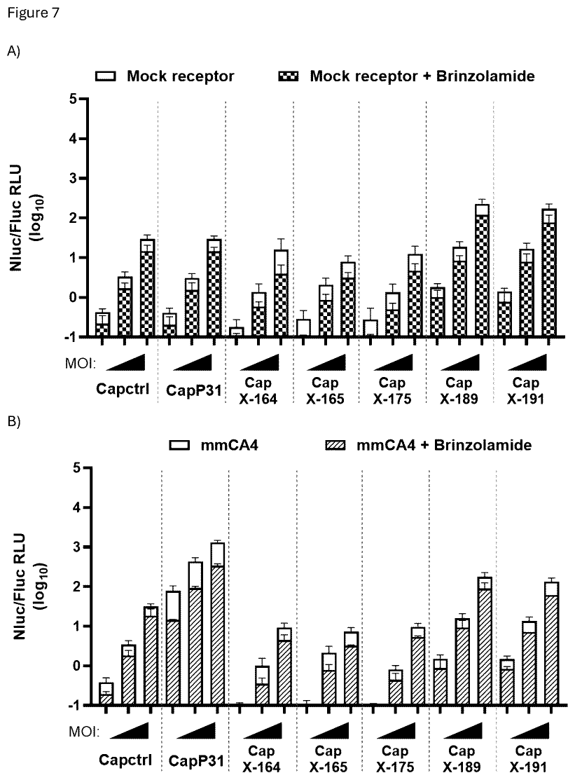

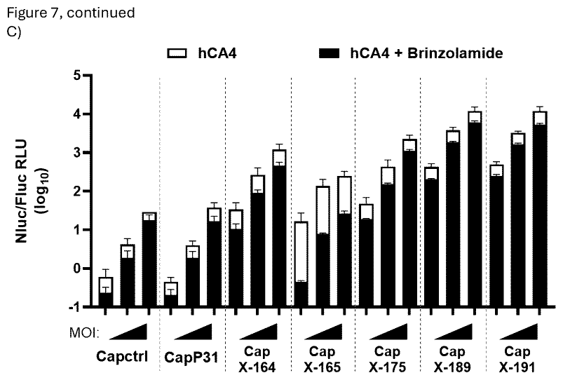

Similar to previous findings, several variants of Capctrl containing random peptide insertions were selectively enriched in hCA4-overexpressing cells (FIG. 3B). Analyzing the top 10 to 100 hCA4-interacting peptide hits, a conserved 7-mer peptide motif XXGLRXL (SEQ ID NO: 305)) was identified, resembling the selected capsid hits from the initial screening campaign (FIG. 2C and FIG. 3C). Sequence alignment of peptide hits from both the first and second campaigns further validated the presence of this motif (FIG. 4A). Phylogenetic analysis of the total hCA4-engaging peptide hits demonstrated clustering around the XXGLRXL (SEQ ID NO: 305) motif, with a secondary, less prominent cluster characterized by the DGPWVNXPR (SEQ ID NO: 303) sequence (FIG. 4B). The recurrence of rAAV capsids displaying highly similar peptide sequences, selectively enriched due to hCA4-targeting strategies across two independent experiments highlights the potential functional significance of the motif sequence in mediating interactions with human CA4. Additionally, this finding underscores the robustness of the capsid library screening methodology.

Subsequently, the top ˜30 hCA4 engager peptide variants of the first in vitro screening were individually cloned into an rAAV9 variant sequence within the pCapX backbone (FIG. 2C). Each pCapX hit was then co-transfected with the pTransgene nano-luciferase (Nluc) reporter into insect cells. Following Bac Rep transactivation, viable rAAV particles were produced, each displaying a novel capsid variant (rAAVCapX) (FIG. 5A).

To confirm and score the engagement of these novel capsid variants with the hCA4 transmembrane receptor, a single-plex hit discovery assay was developed. This assay is based on the principle that exogenous expression of the co-opted transmembrane receptor in mammalian cells selectively improves the potency of rAAV with an engineered capsid that engages with the receptor, compared to naïve AAV9, at a low multiplicity of infection (MOI) (Shay et al., 2024, doi: 10.1126/sciadv.adg6618). In this assay, the CA4 or a mock transmembrane receptor was transiently overexpressed in HEK293T cells via pOX transfection. The pOX plasmid also expressed equimolar mScarlet and Firefly luciferase to represent transfection efficiency. After transfection, each AAVCapX was added, and the cells were assessed for Nluc expression. AAVCapX variants that can co-opthCA4 as an entry receptor are expected to show increased transduction potency compared to those that do not engage with the receptor (FIG. 5B).

The transient overexpression of murine CA4 (mmCA4) in target cells improved the potency of an rAAV9 variant displaying the P31 peptide (Cap9P31), a known mmCA4 engager (Shay et al., Science Advances (2023), 9; Zhang et al., PLOS Pathog (2024) 20 (2)), by approximately ˜10-fold, thereby validating the assay (FIG. 5C). Several rAAVCapX variants displaying the hCA4 peptide hits were confirmed (FIG. 5C) and re-confirmed (FIG. 5D) to engage with hCA4 overexpression, showing a ˜10-100-fold increase in potency. Intriguingly, the CA4 engagement of rAAVCapX hits was found to be highly species-restricted (FIGS. 5C and D), despite the relatively high similarity (˜86% at the protein level) between human and macaque CA4 (mfCA4). This suggests that very precise and delicate interactions are required between the evolved/engineered capsid variant and the newly co-opted transmembrane receptor.

### 3. Example 3: Dose-Dependent and Docking-Site Validation of AAVCapX Interaction with the hCA4 Receptor

### 3.1 Methods

3.1.1. Production of rAAVCapX Variants

Each of the top-selected rAAVCapX variants was cloned into a pCapX plasmid suitable for AAV production in Sf+ cells when co-transfected with a pTransgene plasmid containing nano-luciferase (NLuc) under a ubiquitous promoter (CBh). Using this method, each capsid variant was produced at a small scale. Specifically, pCapX and pTransgene DNA were transfected into 1.5×10\hat{ }7 ExpresSF+ cells using Cellfectin II transfection reagent (ThermoFisher). Seventy-two hours post-transfection, fresh baculovirus expressing AAV2 Replicase was inoculated into the cells at 1% of the final volume (40 ml). Seventy-two hours post-baculovirus infection, the rAAV batches were harvested by adding 10x lysis buffer (1.5 M NaCl, 0.5 M Tris-HCl, 1 mM MgCl2, 10% Triton X-100, pH 8.5) and lysed for 1 hour at 28° C. Genomic DNA was digested by benzonase treatment for 1 hour at 37° C. Cell debris was removed by centrifugation for 15 minutes at 1900×g, after which the supernatant containing the rAAVCapX capsid variants was stored at 4° C.

rAAVCapX capsids were purified from crude lysates using a batch binding protocol involving AAVx affinity resin (ThermoFisher). Briefly, crude cell lysates were added to resin washed with 0.2 M HPO4 buffer (pH 7.5). The samples were incubated for 2 hours at room temperature with gentle mixing. After binding, the resin was washed with 0.2 M HPO4 buffer (pH 7.5), and the bound vectors were eluted by adding 0.2 M Glycine (pH 2.5). The pH of the eluate was immediately neutralized by adding 0.5 M Tris-HCl (pH 8.5). The purified rAAVCapX variant capsids were stored at −20° C.

### 3.1.2. Brinzolamide Competition Assay

HEK293T cells were reverse transfected with plasmids encoding either hCA4, mScarlet, and NLuc reporters or mScarlet and firefly luciferase (FLuc) reporters with a mock receptor. Two days post-transfection, cells were co-incubated with Brinzolamide at a concentration of 100 nM during transduction with each capsid variant. The transduction was performed using vector MOIs of 5E+2, 5E+3, or 5E+4 GC/cell. Forty-eight hours after transduction, cells were lysed, and FLuc and NLuc activities were measured using the Nano-Glo Dual-Luciferase Reporter Assay System. The enrichment score of each capsid variant in the hCA4-transfected cells was calculated by dividing the NLuc readout in hCA4-transfected cells by the NLuc readout in cells transfected with an unrelated receptor. Additionally, a competitive inhibitor binding to the hCA4 receptor was tested. All experiments were performed in triplicate with n=2.

### 3.2 Results

To validate the interaction between the co-opted transmembrane receptor and the engineered rAAV capsid, a dose-dependent, multiplicity of infection (MOI)-based rAAV transduction study was conducted. In this study, rAAVCapX capsid variants with the highest enrichment scores were selected for individual production on a larger scale, with the goal of packaging the Nluc reporter. Each rAAVCapX capsid variant was applied in a dose-dependent manner to HEK293T cells that had been transfected with equimolar amounts of either the pox mock receptor, mmCA4, or hCA4. The expression of the Nluc reporter, normalized to the Fluc expression from the transfected pOX, was measured to assess the transduction potency of the respective rAAV capsids.

As expected, only rAAVCapP31 showed a dose-dependent enhancement in transduction potency in cells overexpressing mmCA4 (FIG. 6A). The enhancement associated with the co-opted receptor was more pronounced at lower MOIs, likely due to the increased general transduction efficiency at higher MOIs via the native AAV receptor, thereby masking the specific effect of the co-opted receptor. Intriguingly, the selected rAAVCapX capsid variants exhibited similar behavior to rAAVCapP31, but only in cells equimolarly overexpressing hCA4 (FIG. 6B). This finding not only confirms the species specificity of the rAAVCapX capsid variants towards the human CA4 ortholog but also suggests that both rAAVCapP31 and the rAAVCapX capsid variants may employ similar mechanisms when using CA4 to cross the blood-brain barrier (BBB).

To further investigate this possibility, a competitive binding assay with brinzolamide, a pan-CA4 small-molecule inhibitor, was performed. Brinzolamide has been shown to competitively block the docking of rAAV9 displaying the P31 peptide (Shay et al., 2024, supra), highlighting the importance of the docking site in mediating the capsid's specific behavior. In our observations, brinzolamide slightly reduced the transduction efficiency of all rAAV capsids, even in cells overexpressing the mock receptor (FIG. 7A), suggesting that the compound, at a dose of 100 nM, might have a general impact on the host cells. Nevertheless, 100 nM brinzolamide clearly out-competed rAAVCapP31 transduction in target cells overexpressing mmCA4 (FIG. 7B), particularly at low MOIs. Surprisingly, among the hCA4 engagers, only rAAVCapX-165 was significantly out-competed by the compound, while only the lowest MOI of rAAVCapX-164 showed significant competition (FIG. 7C). These results suggest that there may be different affinities, or potentially distinct docking sites, among the rAAVCapX variants when interacting with the human CA4 ortholog.

### 4. Example 4: Systemic Intravenous Delivery of hCA4-Interacting rAAVCapX Variants Crosses the BBB and Transduces the Brain In Vivo

### 4.1 Methods

hCA4-interacting rAAVCapX capsid variants are identified an prepared, and a suitable animal model (e.g., mice) is selected for the in vivo experiment. The rAAVCapX capsid variants are administered intravenously via systemic injection, and a control group receiving a non-interacting rAAV capsid variant is included. The subjects are monitored over a specified period for any adverse effects. Post-euthanasia, brain tissue is analyzed for signs of viral transduction using appropriate markers and techniques, such as fluorescence microscopy, PCR, and Western blot.

### 4.2 Results

It is expected that the hCA4-interacting rAAVCapX capsid variants will successfully cross the BBB due to their interaction with hCA4, resulting in significant transduction in the brain tissue of treated animals compared to the control group. Minimal or no transduction is anticipated in the brains of animals receiving the non-interacting rAAV capsid variant, confirming the specificity of the hCA4 interaction. This experiment aims to demonstrate the potential of using hCA4-interacting rAAV capsid variants for targeted gene delivery across the BBB, paving the way for potential therapeutic applications in treating neurological diseases.

### 5. Example 5: HCA4-Interacting rAAVCapX Capsid Variants can Cross the Human BBB as Assessed Using a Human BBB In Vitro Model

### 5.1 Methods

hCA4-interacting rAAVCapX capsid variants are identified and prepared. A human BBB transwell model is constructed by seeding hCMEC/D3 cells at 2×10\hat{ }5 cells/cm² on the top side of a 0.4-μM PET membrane pre-coated with collagen, and human primary astrocytes at 6×10\hat{ }4 cells/cm² on the bottom side of the membrane pre-coated with poly-l-lysine. This model is incubated for 24 hours, after which the hCMEC/D3 cells are seeded on the top side and the culture is maintained with media changes every 2-3 days for 11 days. Heparin sulfate is omitted from the media to avoid interference with rAAV association. The hCA4-interacting rAAVCapX capsid variants are introduced to the top chamber, with a control group exposed to non-hCA4 associating rAAV capsid variants. Transduction efficiency across the BBB model is measured using PCR, immunofluorescence, and electron microscopy to detect viral presence and activity.

### 5.2 Results

It is expected that the hCA4-interacting rAAVCapX capsid variants will demonstrate a significantly higher rate of crossing the human BBB in the transwell model compared to the non-CA4 interacting rAAV capsid variants. This successful transduction will be evidenced by increased viral presence and activity in the bottom chamber of the transwell model, as detected by PCR, immunofluorescence, and electron microscopy. The results will support the potential use of hCA4-interacting rAAV capsid variants for targeted gene delivery across the human BBB, providing insights into developing

1. A recombinant adeno-associated virus (rAAV) capsid comprising at least one binding moiety, wherein the at least one binding moiety confers to the rAAV capsid a phenotype of increased interaction with carbonic anhydrase 4 (CA4) as compared to a control rAAV capsid not comprising the binding moiety.

2. The rAAV capsid according to claim 1, wherein the rAAV capsid presents at least one of the following phenotypes upon systemic administration:
i) a phenotype of increased blood brain barrier (BBB) crossing as compared to a control rAAV capsid not comprising the binding moiety; and/or
ii) a phenotype of increased transduction of cells in the central nervous system (CNS) as compared to a control rAAV capsid not comprising the binding moiety.

3. The rAAV capsid according to claim 1 or 2, wherein at least part of the binding moiety is inserted between two consecutive amino acids of a capsid protein.

4. The rAAV capsid according to any one of claims 1-3, wherein the binding moiety is comprised in a variable region (VR) of a surface loop of the rAAV capsid, preferably in at least one of the VR-I, VR-II, VR-III, VR-IV, VR-V, VR-VI, VR-VII, VR-VIII and/or VR-IX, preferably in the VR-IV and/or VR-VIII, more preferably in the VR-VIII.

5. The rAAV capsid according to any one of claims 1-4, wherein the binding moiety comprises an amino acid insert between two consecutive amino acids present within amino acids 580-595, preferably amino acids 585-590, more preferably amino acids 588 and 589 of an AAV9 capsid protein, or in an analogous position of a capsid protein of an AAV capsid serotype selected from the group comprising: AAV1, AAV2, AAV3A, AAV3B, AAV4, AAV5, AAV6, AAV7, AAV8, AAV10, AAV11, AAV12, AAV13 and AAVrh10, or modified versions thereof, preferably rAAV6, or modified versions thereof.

6. The rAAV capsid according to any one of claims 1-5, wherein the amino acid insert consists of 2, 3, 4, 5, 6, 7, 8, 9, 10, 11, 12, 13, 14, 15 or more amino acids, preferably 5-9 amino acids, more preferably 7 amino acids.

7. The rAAV capsid according to any one of claims 1-6, wherein the binding moiety comprises at least 4, 5, 6, or 7 contiguous amino acids of an amino acid sequence X₁X₂X₃X₄X₅X₆X₇ (SEQ ID NO: 301), wherein:
X₁ is selected from: L, R, V, I, S, D, A, K and P;
X₂ is selected from: H, Q, A, N, E, R, M, G, P, V and W;
X₃ is selected from: G, W, R, S, H, I and V;
X₄ is selected from: L, I, G, F, V and N;
X₅ is selected from: R, Q, S, E, P, A and T;
X₆ is selected from: V, L, R, A, I, S, Q and P, and;
X₇ is selected from: L, I, S, W and R;
optionally wherein the amino acid at position 587 is mutated to D, and/or the amino acid at position 588 is mutated to G.

8. The rAAV capsid according to any one of claims 1-7, wherein:
the binding moiety comprises a consensus sequence AQX₁X₂GX₃X₄X₅L (SEQ ID NO: 302), wherein
X₁ is selected from: L, R, V, I, S, and D;
X₂ is selected from: H, Q, N, E, R, and M;
X₃ is selected from: L and I;
X₄ is selected from: R and Q, and;
X₅ is selected from: V, L and R;
or, wherein the binding moiety comprises a consensus sequence DGPWVNXPR (SEQ ID NO: 303), wherein X is selected from: S, and T;

9. The rAAV capsid according to any one of claims 1-8, wherein the binding moiety comprises or consists of an amino acid sequence selected from SEQ ID NO: 1-168, preferably selected from SEQ ID NO: 1, 2, 12, 26, 28, or 32-36.

10. The rAAV capsid according to any one of claims 1-9, wherein the binding moiety does not compete with brinzolamide for interaction with CA4.

11. The rAAV capsid according to any one of claims 1-10, wherein the rAAV capsid comprises an expression cassette flanked by at least one AAV inverted terminal repeat (ITR), wherein the expression cassette comprises a nucleic acid molecule encoding at least one gene product.

12. A polynucleotide encoding the recombinant adeno-associated virus (rAAV) capsid according to any one of claims 1-11.

13. A host cell comprising a polynucleotide according to claim 12, preferably wherein the polynucleotide is an expression vector for expression of the rAAV capsid.

14. A composition comprising the rAAV capsid according to any one of claims 1-11, preferably wherein the composition is a pharmaceutical composition further comprising at least one pharmaceutically acceptable carrier.

15. The rAAV capsid according to any one of claims 1-11, or the composition of claim 14, for use as a medicament, preferably for use in the treatment of a condition of the central nervous system.

16. The rAAV capsid for use according to claim 15, wherein the rAAV capsid is administered systemically, preferably intravenously or intraarterially.

17. A method of manufacturing an rAAV capsid according to any one of claims 1-11, the method comprising the steps of:
I) culturing a host cell comprising:
i) an expression vector for expression of an rAAV capsid according to any one of claims 1-11, and;
ii) an expression vector encoding an expression cassette flanked by at least one AAV inverted terminal repeat (ITR), comprising a nucleic acid molecule encoding at least one gene product;
said host cell preferably further comprising a nucleotide sequence encoding a parvoviral replication (Rep) protein under conditions such that the rAAV capsid is produced; and,
II) recovery of the rAAV capsid, preferably wherein recovery of the rAAV capsid comprises at least one of: affinity-purification of the rAAV capsid using an immobilized anti-AAV antibody, preferably a single chain camelid antibody or a fragment thereof, or filtration using a filter having a nominal pore size of 30-70 nm.
